SEQUENCE LISTING (97 sequences)

SEQ ID NO: 1 - DNA, 24 bp, synthetic construct
  source 1..24: mol_type = other DNA; note = Primer 1
cctgaggatgccacagtgagacac

SEQ ID NO: 2 - DNA, 28 bp, synthetic construct
  source 1..28: mol_type = other DNA; note = Primer 2
acttctgtctactgtcggactctacagg

SEQ ID NO: 3 - DNA, 22 bp, synthetic construct
  source 1..22: mol_type = other DNA; note = Primer 3.1
gtggcccatttaggcccaactc

SEQ ID NO: 4 - DNA, 24 bp, synthetic construct
  source 1..24: mol_type = other DNA; note = Primer 3.2
ggcccagctttgacttgagaacag

SEQ ID NO: 5 - DNA, 22 bp, synthetic construct
  source 1..22: mol_type = other DNA; note = Primer 3.3
gctgggtctacaggcacaactg

SEQ ID NO: 6 - DNA, 24 bp, synthetic construct
  source 1..24: mol_type = other DNA; note = Probe 1
tctggagggtccactgtgaggcag

SEQ ID NO: 7 - DNA, 24 bp, synthetic construct
  source 1..24: mol_type = other DNA; note = Probe 2
ccatttccaggcctagtgcctgcc

SEQ ID NO: 8 - DNA, 24 bp, synthetic construct
  source 1..24: mol_type = other DNA; note = Probe 3
tgcctgttgaggctgctggcaggc

SEQ ID NO: 9 - DNA, 91 bp, Homo sapiens
  source 1..91: mol_type = genomic DNA; note = chr1_KI270763v1_alt:68038+68128
cctgaggatgccacagtgagacaccatctgggtctggagggtccactgtgaggcagaggctggcctgtagagtccgacagtagacagaagt

SEQ ID NO: 10 - DNA, 91 bp, Homo sapiens
  source 1..91: mol_type = genomic DNA; note = chr17_GL383563v3_alt:4396+4486
cctgaggatgccacagtgagacaccatctgggtctggagggtccactgtgaggcagaggctgacctgtagagtccgacagtagacagaagt

SEQ ID NO: 11 - DNA, 91 bp, Homo sapiens; identical to SEQ ID NO: 10
  source 1..91: mol_type = genomic DNA; note = chr11:130033+130123

SEQ ID NO: 12 - DNA, 91 bp, Homo sapiens; identical to SEQ ID NO: 10
  source 1..91: mol_type = genomic DNA; note = chr17:64396+64486

SEQ ID NO: 13 - DNA, 91 bp, Homo sapiens; identical to SEQ ID NO: 9
  source 1..91: mol_type = genomic DNA; note = chr1:243055930+243056020

SEQ ID NO: 14 - DNA, 91 bp, Homo sapiens; identical to SEQ ID NO: 9
  source 1..91: mol_type = genomic DNA; note = chr1:222475813+222475903

SEQ ID NO: 15 - DNA, 91 bp, Homo sapiens; identical to SEQ ID NO: 10
  source 1..91: mol_type = genomic DNA; note = chr1:728464+728554

SEQ ID NO: 16 - DNA, 91 bp, Homo sapiens; identical to SEQ ID NO: 10
  source 1..91: mol_type = genomic DNA; note = chr1:493558+493648

SEQ ID NO: 17 - DNA, 91 bp, Homo sapiens; identical to SEQ ID NO: 10
  source 1..91: mol_type = genomic DNA; note = chr1:138356+138446

SEQ ID NO: 18 - DNA, 91 bp, Homo sapiens
  source 1..91: mol_type = genomic DNA; note = chr4:119408800+119408890
cctgaggatgccacagtgagacaccatctgggtctgcagggtccactgtgaggcagaggctggcctgtagagtccgacagtagacacaagt

SEQ ID NO: 19 - DNA, 91 bp, Homo sapiens; identical to SEQ ID NO: 9
  source 1..91: mol_type = genomic DNA; note = chr6:146543+146633

SEQ ID NO: 20 - DNA, 91 bp, Homo sapiens; identical to SEQ ID NO: 9
  source 1..91: mol_type = genomic DNA; note = chr7:56377197+56377287

SEQ ID NO: 21 - DNA, 91 bp, Homo sapiens; identical to SEQ ID NO: 9
  source 1..91: mol_type = genomic DNA; note = chr7:55746723+55746813

SEQ ID NO: 22 - DNA, 91 bp, Homo sapiens; identical to SEQ ID NO: 10
  source 1..91: mol_type = genomic DNA; note = chrUn_KI270745v1:20572-20662

SEQ ID NO: 23 - DNA, 91 bp, Homo sapiens; identical to SEQ ID NO: 9
  source 1..91: mol_type = genomic DNA; note = chr20:64288636-64288726

SEQ ID NO: 24 - DNA, 91 bp, Homo sapiens; identical to SEQ ID NO: 9
  source 1..91: mol_type = genomic DNA; note = chr1:227968791-227968881

SEQ ID NO: 25 - DNA, 91 bp, Homo sapiens; identical to SEQ ID NO: 9
  source 1..91: mol_type = genomic DNA; note = chr4:118631693-118631783

SEQ ID NO: 26 - DNA, 91 bp, Homo sapiens; identical to SEQ ID NO: 10
  source 1..91: mol_type = genomic DNA; note = chr5:181325303-181325393

SEQ ID NO: 27 - DNA, 91 bp, Homo sapiens
  source 1..91: mol_type = genomic DNA; note = chr7:128652424-128652514
actgaggatgccacagtgagacaccatctgggtctggagggtccactgtgaggcagaggctggcctgtagagtccgacagtagacagaagt

SEQ ID NO: 28 - DNA, 91 bp, Homo sapiens; identical to SEQ ID NO: 9
  source 1..91: mol_type = genomic DNA; note = chr7:63746664-63746754

SEQ ID NO: 29 - DNA, 91 bp, Homo sapiens; identical to SEQ ID NO: 9
  source 1..91: mol_type = genomic DNA; note = chr7:45808736-45808826

SEQ ID NO: 30 - DNA, 200 bp, Homo sapiens
  source 1..200: mol_type = genomic DNA; note = chr1_KI270763v1_alt:68038+68237
cctgaggatgccacagtgagacaccatctgggtctggagggtccactgtgaggcagaggctggcctgtagagtccgacagtagacagaagttgggcaaaaggctgatttgaggaagttttgggcttcaagagtcagccacgaggcaggcactaggcctggaaatggcctgacagtcatgagttgggcctaaatgggccac

SEQ ID NO: 31 - DNA, 200 bp, Homo sapiens
  source 1..200: mol_type = genomic DNA; note = chr17_GL383563v3_alt:4396+4595
cctgaggatgccacagtgagacaccatctgggtctggagggtccactgtgaggcagaggctgacctgtagagtccgacagtagacagaagttgggcaaaaggctgatttgaggaagttttgggcttcaagagtcagccacgaggcaggcactaggcctggaaatggcctcacagtcatgagttgggcctaaatgggccac

SEQ ID NO: 32 - DNA, 200 bp, Homo sapiens
  source 1..200: mol_type = genomic DNA; note = chr11:130033+130232
cctgaggatgccacagtgagacaccatctgggtctggagggtccactgtgaggcagaggctgacctgtagagtccgacagtagacagaagttgggcaaaagcctgatttgaggaagttttgggcttcaagagtcagccacgaggcaggcactaggcctggaaatggcctcacagtcatgagttgggcctaaatgggccac

SEQ ID NO: 33 - DNA, 200 bp, Homo sapiens; identical to SEQ ID NO: 31
  source 1..200: mol_type = genomic DNA; note = chr17:64396+64595

SEQ ID NO: 34 - DNA, 200 bp, Homo sapiens; identical to SEQ ID NO: 30
  source 1..200: mol_type = genomic DNA; note = chr1:243055930+243056129

SEQ ID NO: 35 - DNA, 200 bp, Homo sapiens
  source 1..200: mol_type = genomic DNA; note = chr1:728464+728663
cctgaggatgccacagtgagacaccatctgggtctggagggtccactgtgaggcagaggctgacctgtagagtccgacagtagacagaagttgggcaaaagcctgatttgaggaagttttgggcttcaagagtcagccacgaggcaggcactaggcctggaaatggactcacagtcatgagttgggcctaaatgggccac

SEQ ID NO: 36 - DNA, 200 bp, Homo sapiens; identical to SEQ ID NO: 32
  source 1..200: mol_type = genomic DNA; note = chr1:493558+493757

SEQ ID NO: 37 - DNA, 200 bp, Homo sapiens
  source 1..200: mol_type = genomic DNA; note = chr4:119408800+119408999
cctgaggatgccacagtgagacaccatctgggtctgcagggtccactgtgaggcagaggctggcctgtagagtccgacagtagacacaagttgggcaaaaggctgatttgaggaagttttgggcttcaagagtcagccaggaggcaggcactaggcctggaaatggcccgacagtcatgagttgggcctaaatgggccac

SEQ ID NO: 38 - DNA, 200 bp, Homo sapiens
  source 1..200: mol_type = genomic DNA; note = chr6:146543+146742
cctgaggatgccacagtgagacaccatctgggtctggagggtccactgtgaggcagaggctggcctgtagagtccgacagtagacagaagttgggcaaaaggctgatttgaggaagttttgggcttcaacagtcagccacgaggcagacactaggcctggaaatagcccgacagtcatgagttgggcctaaatgggccac

SEQ ID NO: 39 - DNA, 200 bp, Homo sapiens
  source 1..200: mol_type = genomic DNA; note = chr7:56830262+56830461
cctgaggatgccacagtgagacaccatctgggtctggagggtccactgtgaggcagaggctggcctgtagagtccaacagtagacagaagttgagcaaaaggctaatttcaggaagttttgggcttcaagagtcagccaggaggcaggcactaggcctggaaatggcccgacagtcatgagttgggcctaaatgggccac

SEQ ID NO: 40 - DNA, 200 bp, Homo sapiens
  source 1..200: mol_type = genomic DNA; note = chr7:56377197+56377396
cctgaggatgccacagtgagacaccatctgggtctggagggtccactgtgaggcagaggctggcctgtagagtccgacagtagacagaagttgggcaaaaggctgatttgaggaagttttgggcttcaagagtcagccaggaggcaggcactaggcctggaaatggcccgacagtcatgagttgggcctaaatgggccac

SEQ ID NO: 41 - DNA, 200 bp, Homo sapiens; identical to SEQ ID NO: 40
  source 1..200: mol_type = genomic DNA; note = chr7:55746723+55746922

SEQ ID NO: 42 - DNA, 200 bp, Homo sapiens
  source 1..200: mol_type = genomic DNA; note = chr7:51391393+51391592
cctgaggatgccacagtgagacaccatctgggtctggagggtccactgtgaggcagaggctggcctgtagagtccaacagtaggcagaggttgggcaaaaggctgattcgaggaagttttgggcttcaagagtcagccaggaggcaggcgctaggcctggaaatggcccgacagtcatgagttgggcctaaatgggccac

SEQ ID NO: 43 - DNA, 200 bp, Homo sapiens; identical to SEQ ID NO: 31
  source 1..200: mol_type = genomic DNA; note = chrUn_KI270745v1:20463-20662

SEQ ID NO: 44 - DNA, 200 bp, Homo sapiens
  source 1..200: mol_type = genomic DNA; note = chr16:90167272-90167471
cctgaggatgccacagtgagacaccatctgagtctggagggtccactgtgaggcagaggctggcctgtagagtccaacagtagacagaagttgggcaaaaggctgatttgaggaagttttgggcttcaagagtcagccacgaggcaggcactaggcctggaaatggcctcacagtcatgagttgggcctaaatgggccac

SEQ ID NO: 45 - DNA, 200 bp, Homo sapiens; identical to SEQ ID NO: 38
  source 1..200: mol_type = genomic DNA; note = chr20:64288527-64288726

SEQ ID NO: 46 - DNA, 200 bp, Homo sapiens
  source 1..200: mol_type = genomic DNA; note = chr1:227968682-227968881
cctgaggatgccacagtgagacaccatctgggtctggagggtccactgtgaggcagaggctggcctgtagagtccgacagtagacagaagttgggcaaaaggctgatttgaggaagttttgggcttcaagagtcagccacgaggcaggcactaggcctggaaatggcccgacagtcatgagttgggcctaaatgggccac

SEQ ID NO: 47 - DNA, 200 bp, Homo sapiens; identical to SEQ ID NO: 32
  source 1..200: mol_type = genomic DNA; note = chr5:181325194-181325393

SEQ ID NO: 48 - DNA, 200 bp, Homo sapiens
  source 1..200: mol_type = genomic DNA; note = chr7:128652315-128652514
actgaggatgccacagtgagacaccatctgggtctggagggtccactgtgaggcagaggctggcctgtagagtccgacagtagacagaagttgggcaaaaggctgatttgaggaagttttgggcttcaagagtcagccacgaggcaggcactaggcctggaaatggcccgacagtcatgagttgggcctaaatgggccac

SEQ ID NO: 49 - DNA, 200 bp, Homo sapiens
  source 1..200: mol_type = genomic DNA; note = chr7:63746555-63746754
cctgaggatgccacagtgagacaccatctgggtctggagggtccactgtgaggcagaggctggcctgtagagtccgacagtagacagaagttgagcaaaaggctaatttcaggaagttttgggcttcaagagtcagccaggaggcaggcactaggcctggaaatggcccgacagtcatgagttgggcctaaatgggccac

SEQ ID NO: 50 - DNA, 200 bp, Homo sapiens; identical to SEQ ID NO: 40
  source 1..200: mol_type = genomic DNA; note = chr7:45808627-45808826

SEQ ID NO: 51 - DNA, 352 bp, Homo sapiens
  source 1..352: mol_type = genomic DNA; note = chr1_KI270763v1_alt:68038+68390
cctgaggatgccacagtgagacaccatctgggtctggagggtccactgtgaggcagaggctggcctgtagagtccgacagtagacagaagttgggcaaaaggctgatttgaggaagttttgggcttcaagagtcagccacgaggcaggcactaggcctggaaatggcctgacagtcatgagttgggcctaaatgggccactgtgagggaggagctgtgcctgttgaggctgctggcaggcaggcagaaattcggcctggggcagctgccatgaggcaagagctgggcctggaaaaagcccctgggaggcaagagcagggcctgcagaggctgttctcaagtcaagctgggcc

SEQ ID NO: 52 - DNA, 352 bp, Homo sapiens
  source 1..352: mol_type = genomic DNA; note = chr17_GL383563v3_alt:4396+4748
cctgaggatgccacagtgagacaccatctgggtctggagggtccactgtgaggcagaggctgacctgtagagtccgacagtagacagaagttgggcaaaggctgatttgaggaagttttgggcttcaagagtcagccacgaggcaggcactaggcctggaaatggcctcacagtcatgagttgggcctaaatgggccactgtgagggaggagctgtgcctgttgaggctgctggcaggcaggcagaaatttggcctggggcagccgccatgaggcaagagctgggcctggaaaaagcccctgggaggcaagagcagggcctgcagaggctgttctcaagtcaaagctgggcc

SEQ ID NO: 53 - DNA, 353 bp, Homo sapiens
  source 1..353: mol_type = genomic DNA; note = chr11:130033+130385
cctgaggatgccacagtgagacaccatctgggtctggagggtccactgtgaggcagaggctgacctgtagagtccgacagtagacagaagttgggcaaaagcctgatttgaggaagttttgggcttcaagagtcagccacgaggcaggcactaggcctggaaatggcctcacagtcatgagttgggcctaaatgggccactgtgagggaggagctgtgcctgttgaggctgctggcaggcaggcagaaatttggcctggggcagctgccatgaggcaagagctgggcctggaaaaagcccctgggaggcaagagcagggcctgcagaggctgttctcaagtcaaagctgggcc

SEQ ID NO: 54 - DNA, 353 bp, Homo sapiens
  source 1..353: mol_type = genomic DNA; note = chr17:64396+64748
cctgaggatgccacagtgagacaccatctgggtctggagggtccactgtgaggcagaggctgacctgtagagtccgacagtagacagaagttgggcaaaaggctgatttgaggaagttttgggcttcaagagtcagccacgaggcaggcactaggcctggaaatggcctcacagtcatgagttgggcctaaatgggccactgtgagggaggagctgtgcctgttgaggctgctggcaggcaggcagaaatttggcctggggcagccgccatgaggcaagagctgggcctggaaaaagcccctgggaggcaagagcagggcctgcagaggctgttctcaagtcaaagctgggcc

SEQ ID NO: 55 - DNA, 353 bp, Homo sapiens
  source 1..353: mol_type = genomic DNA; note = chr1:243055930+243056282
cctgaggatgccacagtgagacaccatctgggtctggagggtccactgtgaggcagaggctggcctgtagagtccgacagtagacagaagttgggcaaaaggctgatttgaggaagttttgggcttcaagagtcagccacgaggcaggcactaggcctggaaatggcctgacagtcatgagttgggcctaaatgggccactgtgagggaggagctgtgcctgttgaggctgctggcaggcaggcagaaattcggcctggggcagctgccatgaggcaagagctgggcctggaaaaagcccctgggaggcaagagcagggcctgcagaggctgttctcaagtcaaagctgggcc

SEQ ID NO: 56 - DNA, 353 bp, Homo sapiens
  source 1..353: mol_type = genomic DNA; note = chr1:222475813+222476165
cctgaggatgccacagtgagacaccatctgggtctggagggtccactgtgaggcagaggctggcctgtagagtccgacagtagacagaagttgggcaaaaggctgatttgaggaagttttgggcttcaagagtcagccacgaggcaggcactaggcctggaaatggcccgacagtcatgagttgggactaaatgggccactgtgagggaggagctgtgcctgttgaggctgctggcaggcaagcagaaatttggcctcgggcagctgccatgaggcaagagctgggcctggaaaaagcccctgggaggcaagagcagggcctgcagaggctgttctcaagtcaaagctgggcc

SEQ ID NO: 57 - DNA, 353 bp, Homo sapiens
  source 1..353: mol_type = genomic DNA; note = chr1:728464+728816
cctgaggatgccacagtgagacaccatctgggtctggagggtccactgtgaggcagaggctgacctgtagagtccgacagtagacagaagttgggcaaaagcctgatttgaggaagttttgggcttcaagagtcagccacgaggcaggcactaggcctggaaatggactcacagtcatgagttgggcctaaatgggccactgtgagggaggagctgtgcctgttgaggctgctggcaggcaggcagaaatttggcctggggcagctgccatgaggcaagagctgggcctggaaaaagcccctgggaggcaagagcagggcctgcagaggctgttctcaagtcaaagctgggcc

SEQ ID NO: 58 - DNA, 353 bp, Homo sapiens; identical to SEQ ID NO: 53
  source 1..353: mol_type = genomic DNA; note = chr1:493558+493910

SEQ ID NO: 59 - DNA, 352 bp, Homo sapiens
  source 1..352: mol_type = genomic DNA; note = chr1:138356+138708
cctgaggatgccacagtgagacaccatctgggtctggagggtccactgtgaggcagaggctgacctgtagagtccgacagtagacagaagttgggcaaaagcctgatttgaggaagttttgggcttcaagagtcagccacgaggcaggcactaggcctggaaatggcctcacagtcataagttgggcctaaatgggccactgtgagggaggagctgtgcctgttgaggctgctggcaggcaggcagaaatttggcctggggcagctgccatgaggcaagagctgggcctggaaaaagccctgggaggcaagagcagggcctgcagaggctgttctcaagtcaaagctgggcc

SEQ ID NO: 60 - DNA, 352 bp, Homo sapiens
  source 1..352: mol_type = genomic DNA; note = chr4:119408800+119409151
cctgaggatgccacagtgagacaccatctgggtctgcagggtccactgtgaggcagaggctggcctgtagagtccgacagtagacacaagttgggcaaaaggctgatttgaggaagttttgggcttcaagagtcagccaggaggcaggcactaggcctggaaatggcccgacagtcatgagttgggcctaaatgggccaccgtgagggaggagctgtgcctgttgaggctgctggcaggcaggaagaaatttggcctgaggcagctgccatgaggcaagagctgggcttggaaaagcccctgggaggcaagagcaggccctgcagaggctgttctcaagtcaaagctgggcc

SEQ ID NO: 61 - DNA, 352 bp, Homo sapiens
  source 1..352: mol_type = genomic DNA; note = chr7:56830262+56830613
cctgaggatgccacagtgagacaccatctgggtctggagggtccactgtgaggcagaggctggcctgtagagtccaacagtagacagaagttgagcaaaaggctaatttcaggaagttttgggcttcaagagtcagccaggaggcaggcactaggcctggaaatggcccgacagtcatgagttgggcctaaatgggccactgtgagggaggagctgtgcctgttgaggctgctggcaggcaggcagaaacttggcctggggcagctgccatgagacaagagctgggcctggagaagcccctgagaggtaagagcagggcctgcagaggctgttctcaagtcaaagctgggcc

SEQ ID NO: 62 - DNA, 352 bp, Homo sapiens
  source 1..352: mol_type = genomic DNA; note = chr7:56377197+56377548
cctgaggatgccacagtgagacaccatctgggtctggagggtccactgtgaggcagaggctggcctgtagagtccgacagtagacagaagttgggcaaaaggctgatttgaggaagttttgggcttcaagagtcagccaggaggcaggcactaggcctggaaatggcccgacagtcatgagttgggcctaaatgggccactgtgagggaggagctgtgcctgttgaggctgctggcaggcaggcagaaacttggcctggggcagccgccatgaggcaagagctgggcctggagaagcccctgggaggcaagagcacggcctgcagaggctgttctcaagtcaaagctgggcc

SEQ ID NO: 63 - DNA, 352 bp, Homo sapiens; identical to SEQ ID NO: 62
  source 1..352: mol_type = genomic DNA; note = chr7:55746723+55747074

SEQ ID NO: 64 - DNA, 353 bp, Homo sapiens
  source 1..353: mol_type = genomic DNA; note = chrUn_KI270745v1:20310-20662
cctgaggatgccacagtgagacaccatctgggtctggagggtccactgtgaggcagaggctgacctgtagagtccgacagtagacagaagttgggcaaaaggctgatttgaggaagttttgggcttcaagagtcagccacgaggcaggcactaggcctggaaatggcctcacagtcatgagttgggcctaaatgggccactgtgagggaggagctgtgcctgttgaggctgctggcaggcaggcagaaatttggcctggggcagctgccatgaggcaagagctgggcctggaaaaagcccctgggaggcaagagcagggcctgcagaggctgttctcaagtcaaagctgggcc

SEQ ID NO: 65 - DNA, 352 bp, Homo sapiens
  source 1..352: mol_type = genomic DNA; note = chr16:90167120-90167471
cctgaggatgccacagtgagacaccatctgagtctggagggtccactgtgaggcagaggctggcctgtagagtccaacagtagacagaagttgggcaaaaggctgatttgaggaagttttgggcttcaagagtcagccacgaggcaggcactaggcctggaaatggcctcacagtcatgagttgggcctaaatgggccactgtgagggaggagctgtgcctgttgaggctgctggcaggcaggcagaaatttggcctgggcagctgccacgaggcaagagctgggcctggaaaaagcccctgggaggcaagagcagggcctgcagaggctgttctcaagtcaaagctgggcc

SEQ ID NO: 66 - DNA, 353 bp, Homo sapiens
  source 1..353: mol_type = genomic DNA; note = chr1:227968529-227968881
cctgaggatgccacagtgagacaccatctgggtctggagggtccactgtgaggcagaggctggcctgtagagtccgacagtagacagaagttgggcaaaaggctgatttgaggaagttttgggcttcaagagtcagccacgaggcaggcactaggcctggaaatggcccgacagtcatgagttgggcctaaatgggccactgtgagggaggagctgtgcctgttgaggctgctggcaggcaggcagaaatttggcctggggcagctgccatgaggcaggagctgggtctggaaaaagcccctgggaggcaagagcagggcctgcagaggctgttctcaagtcaaagctgggcc

SEQ ID NO: 67 - DNA, 356 bp, Homo sapiens
  source 1..356: mol_type = genomic DNA; note = chr4:118631428-118631783
cctgaggatgccacagtgagacaccatctgggtctggagggtccactgtgaggcagaggctggcctgtagagtccgacagtagacagaagttaggcaaaaggctgatttgaggaagttttgggcttcaagaaagagtcagccaggaggcaggcactaggcctggaaatggcccgacagtcatgagtggggcctaaatgggccactgtgagggaggagctgtgcctgttgaggctgctggcaggcaggaagaaatttggcctggggcagctgccatgaggcaagagctgggcttggaaaagcccctgggaggcaagagcagggcctgcagaggctgttctcaagtcaaagctgggcc

SEQ ID NO: 68 - DNA, 353 bp, Homo sapiens; identical to SEQ ID NO: 53
  source 1..353: mol_type = genomic DNA; note = chr5:181325041-181325393

SEQ ID NO: 69 - DNA, 353 bp, Homo sapiens
  source 1..353: mol_type = genomic DNA; note = chr7:128652162-128652514
actgaggatgccacagtgagacaccatctgggtctggagggtccactgtgaggcagaggctggcctgtagagtccgacagtagacagaagttgggcaaaaggctgatttgaggaagttttgggcttcaagagtcagccacgaggcaggcactaggcctggaaatggcccgacagtcatgagttgggcctaaatgggccactgtgagggaggagctatgcctgttgaggctgctggcaggcaggcagaaatttggcctggggcagctgccatgaggcaagagctgggcctggaaaaagcccctgggaggcaagagcagggcctgcagaggctgttctcaagtcaaagctgggcc

SEQ ID NO: 70 - DNA, 352 bp, Homo sapiens
  source 1..352: mol_type = genomic DNA; note = chr7:63746403-63746754
cctgaggatgccacagtgagacaccatctgggtctggagggtccactgtgaggcagaggctggcctgtagagtccgacagtagacagaagttgagcaaaaggctaatttcaggaagttttgggcttcaagagtcagccaggaggcaggcactaggcctggaaatggcccgacagtcatgagttgggcctaaatgggccactgtgagggaggagctgtgcctgttgaggctgctggcaggcaggcagaaacttggcctggagcagcttccatgagacaagagctgggcctggagaagcccctgagaggtaagaggagggcctgcagaggctgttctcaagtcaaagctgggcc

SEQ ID NO: 71 - DNA, 352 bp, Homo sapiens
  source 1..352: mol_type = genomic DNA; note = chr7:45808475-45808826
cctgaggatgccacagtgagacaccatctgggtctggagggtccactgtgaggcagaggctggcctgtagagtccgacagtagacagaagttgggcaaaaggctgatttgaggaagttttgggcttcaagagtcagccaggaggcaggcactaggcctggaaatggcccgacagtcatgagttgggcctaaatgggccactgtgagggaagagctgtgcctgttgaggctgctggcaggcaggcagaaacttggcctggggcagccgccatgaggcaagagctgggcctggagaagcccctgggaggcaagagcagggcctgcagaggctgttctcaagtcaaagctgggcc

SEQ ID NO: 72 - DNA, 496 bp, Homo sapiens
  source 1..496: mol_type = genomic DNA; note = chr1_KI270763v1_alt:68038+68533
cctgaggatgccacagtgagacaccatctgggtctggagggtccactgtgaggcagaggctggcctgtagagtccgacagtagacagaagttgggcaaaaggctgatttgaggaagttttgggcttcaagagtcagccacgaggcaggcactaggcctggaaatggcctgacagtcatgagttgggcctaaatgggccactgtgagggaggagctgtgcctgttgaggctgctggcaggcaggcagaaattcggcctggggcagctgccatgaggcaagagctgggcctggaaaaagcccctgggaggcaagagcagggcctgcagaggctgttctcaagtcaaagctgggccggttcatgccaccgggaagcagaaggtgggcctggagagtttgacttgaggaagttttgggcctacattggccgccatgagctggacaggaactgggccaaaaaaggctgttgtgaggcagcagttgtgcctgtagacccagc

SEQ ID NO: 73 - DNA, 496 bp, Homo sapiens
  source 1..496: mol_type = genomic DNA; note = chr17_GL383563v3_alt:4396+4891
cctgaggatgccacagtgagacaccatctgggtctggagggtccactgtgaggcagaggctgacctgtagagtccgacagtagacagaagttgggcaaaaggctgatttgaggaagttttgggcttcaagagtcagccacgaggcaggcactaggcctggaaatggcctcacagtcatgagttgggcctaaatgggccactgtgagggaggagctgtgcctgttgaggctgctggcaggcaggcagaaatttggcctggggcagccgccatgaggcaagagctgggcctggaaaaagcccctgggaggcaagagcagggcctgcagaggctgttctcaagtcaaagctgggcctgttgatgccaccgggaagcagaaggtgggcctggagagtttgacttgaggaagttttgggcctacattggccgccatgagctggacaggaactgggccaaaaaaggctgttgtgaggcagcagttgtgcctgtagacccagc

SEQ ID NO: 74 - DNA, 496 bp, Homo sapiens
  source 1..496: mol_type = genomic DNA; note = chr11:130033+130528
cctgaggatgccacagtgagacaccatctgggtctggagggtccactgtgaggcagaggctgacctgtagagtccgacagtagacagaagttgggcaaaagcctgatttgaggaagttttgggcttcaagagtcagccacgaggcaggcactaggcctggaaatggcctcacagtcatgagttgggcctaaatgggccactgtgagggaggagctgtgcctgttgaggctgctggcaggcaggcagaaatttggcctggggcagctgccatgaggcaagagctgggcctggaaaaagcccctgggaggcaagagcagggcctgcagaggctgttctcaagtcaaagctgggcctgttgatgccaccgggaagcagaaggtgggcctggagagtttgacttgaggaagttttgggcctacattggccgccatgagctggacaggaactgggccaaaaaaggctgttgtgaggcagcagttgtgcctgtagacccagc

SEQ ID NO: 75 - DNA, 496 bp, Homo sapiens; identical to SEQ ID NO: 73
  source 1..496: mol_type = genomic DNA; note = chr17:64396+64891

SEQ ID NO: 76 - DNA, 496 bp, Homo sapiens; identical to SEQ ID NO: 72
  source 1..496: mol_type = genomic DNA; note = chr1:243055930+243056425

SEQ ID NO: 77 - DNA, 496 bp, Homo sapiens
  source 1..496: mol_type = genomic DNA; note = chr1:222475813+222476308
cctgaggatgccacagtgagacaccatctgggtctggagggtccactgtgaggcagaggctggcctgtagagtccgacagtagacagaagttgggcaaaaggctgatttgaggaagttttgggcttcaagagtcagccacgaggcaggcactaggcctggaaatggcccgacagtcatgagttgggactaaatgggccactgtgagggaggagctgtgcctgttgaggctgctggcaggcaagcagaaatttggcctcgggcagctgccatgaggcaagagctgggcctggaaaaagcccctgggaggcaagagcagggcctgcagaggctgttctcaagtcaaagctgggcctgttcatgccaccgggaagcagaaggtgggcctggagagtttgacttgaggaagttttgggcctacattggccgccatgagctggacaggaactgggccaaaaaaggctgttgtgaggcagcagttgtgcctgtagacccagc

SEQ ID NO: 78 - DNA, 496 bp, Homo sapiens
  source 1..496: mol_type = genomic DNA; note = chr1:728464+728959
cctgaggatgccacagtgagacaccatctgggtctggagggtccactgtgaggcagaggctgacctgtagagtccgacagtagacagaagttgggcaaaagcctgatttgaggaagttttgggcttcaagagtcagccacgaggcaggcactaggcctggaaatggactcacagtcatgagttgggcctaaatgggccactgtgagggaggagctgtgcctgttgaggctgctggcaggcaggcagaaatttggcctggggcagctgccatgaggcaagagctgggcctggaaaaagcccctgggaggcaagagcagggcctgcagaggctgttctcaagtcaaagctgggcctgttgatgccaccgggaagcagaaggtgggcctggagagtttgacttgaggaagttttgggcctacattggccgccatgagctggacaggaactgggccaaaaaaggctgttgtgaggcagcagttgtgcctgtagacccagc

SEQ ID NO: 79 - DNA, 496 bp, Homo sapiens; identical to SEQ ID NO: 74
  source 1..496: mol_type = genomic DNA; note = chr1:493558+494053

SEQ ID NO: 80 - DNA, 496 bp, Homo sapiens
  source 1..496: mol_type = genomic DNA; note = chr1:138356+138851
cctgaggatgccacagtgagacaccatctgggtctggagggtccactgtgaggcagaggctgacctgtagagtccgacagtagacagaagttgggcaaaagcctgatttgaggaagttttgggcttcaagagtcagccacgaggcaggcactaggcctggaaatggcctcacagtcataagttgggcctaaatgggccactgtgagggaggagctgtgcctgttgaggctgctggcaggcaggcagaaatttggcctggggcagctgccatgaggcaagagctgggcctggaaaaagcccctgggaggcaagagcagggcctgcagaggctgttctcaagtcaaagctgggcctgttgatgccaccgggaagcagaaggtgggcctggagagtttgacttgaggaagttttgggcctacattggccgccattagctggacaggaactgggccaaaaaaggctgttgtgaggcagcagttgtgcctgtagacccagc

SEQ ID NO: 81 - DNA, 495 bp, Homo sapiens
  source 1..495: mol_type = genomic DNA; note = chr4:119408800+119409294
cctgaggatgccacagtgagacaccatctgggtctgcagggtccactgtgaggcagaggctggcctgtagagtccgacagtagacacaagttgggcaaaaggctgatttgaggaagttttgggcttcaagagtcagccaggaggcaggcactaggcctggaaatggcccgacagtcatgagttgggcctaaatgggccaccgtgagggaggagctgtgcctgttgaggctgctggcaggcaggaagaaatttggcctgaggcagctgccatgaggcaagagctgggcttggaaaagcccctgggaggcaagagcaggccctgcagaggctgttctcaagtcaaagctgggcctgttcatgccaccgggaagcagaaggtgggcctggagagtatgacttgaggaagttttgggcctacattggctgccatgagctggacaggaactgggccaaaaaaggctgttgtgaggcagcagttgtgcctgtagactcagc

SEQ ID NO: 82 - DNA, 509 bp, Homo sapiens
  source 1..509: mol_type = genomic DNA; note = chr7:56830262+56830770
cctgaggatgccacagtgagacaccatctgggtctggagggtccactgtgaggcagaggctggcctgtagagtccaacagtagacagaagttgagcaaaaggctaatttcaggaagttttgggcttcaagagtcagccaggaggcaggcactaggcctggaaatggcccgacagtcatgagttgggcctaaatgggccactgtgagggaggagctgtgcctgttgaggctgctggcaggcaggcagaaacttggcctggggcagctgccatgagacaagagctgggcctggagaagcccctgagaggtaagagcagggcctgcagaggctgttctcaagtcaaagctgggcctgttcatgccaccgggaagcagaaggtgggcctggagagttttacttgaggaagttttgggcctacatttgctgccgtgagctggacaggaactgggccaaaaaaggctgttgtgaggcagctgttatgaggcagcagttgtgcctgtagactcagc

SEQ ID NO: 83 - DNA, 495 bp, Homo sapiens
  source 1..495: mol_type = genomic DNA; note = chr7:56377197+56377691
cctgaggatgccacagtgagacaccatctgggtctggagggtccactgtgaggcagaggctggcctgtagagtccgacagtagacagaagttgggcaaaaggctgatttgaggaagttttgggcttcaagagtcagccaggaggcaggcactaggcctggaaatggcccgacagtcatgagttgggcctaaatgggccactgtgagggaggagctgtgcctgttgaggctgctggcaggcaggcagaaacttggcctggggcagccgccatgaggcaagagctgggcctggagaagcccctgggaggcaagagcacggcctgcagaggctgttctcaagtcaaagctgggcctgttcatgccaccgggaagcagaaggcgggcctggggagtttgacttgaggaagttttgggcctacattggccgctgtgagctggacaggaactgggccaaaaaaggctgttgtgaagcagcagttgtgcctgtagactcagc

SEQ ID NO: 84 - DNA, 495 bp, Homo sapiens
  source 1..495: mol_type = genomic DNA; note = chr7:55746723+55747217
cctgaggatgccacagtgagacaccatctgggtctggagggtccactgtgaggcagaggctggcctgtagagtccgacagtagacagaagttgggcaaaaggctgatttgaggaagttttgggcttcaagagtcagccaggaggcaggcactaggcctggaaatggcccgacagtcatgagttgggcctaaatgggccactgtgagggaggagctgtgcctgttgaggctgctggcaggcaggcagaaacttggcctggggcagccgccatgaggcaagagctgggcctggagaagcccctgggaggcaagagcacggcctgcagaggctgttctcaagtcaaagctgggcctgttcatgccaccgcgaagcggaaggcgggcctggagagtttgacttgaggaagttttgggcctacattggccgctgtgagctggacaggaactgggccaaaaaaggctgttgtgaggcagcagttgtgcctgtagactcagc

SEQ ID NO: 85 - DNA, 495 bp, Homo sapiens
  source 1..495: mol_type = genomic DNA; note = chr7:51391393+51391887
cctgaggatgccacagtgagacaccatctgggtctggagggtccactgtgaggcagaggctggcctgtagagtccaacagtaggcagaggttgggcaaaaggctgattcgaggaagttttgggcttcaagagtcagccaggaggcaggcgctaggcctggaaatggcccgacagtcatgagttgggcctaaatgggccactgtgagggaggagctgtgcctgttgaggctgctggcaggcaggcagaaacttggcctggggcagctgccatgaggcaagagctgggcctggagaagcccctgggaggcaagagcagggcctgcagaggctattctcaagtcaaagctgggcctgttcatgccaccgggaagcagaaggtgggcctggagagtttgacttgaggaagttttgggcctacattggccgccgtgagctggacaggaactgggccaaaaaaggctgttgtgaggcagcagttgtgcctgtagactcagc

SEQ ID NO: 86 - DNA, 496 bp, Homo sapiens
  source 1..496: mol_type = genomic DNA; note = chrUn_KI270745v1:20167-20662
cctgaggatgccacagtgagacaccatctgggtctggagggtccactgtgaggcagaggctgacctgtagagtccgacagtagacagaagttgggcaaaaggctgatttgaggaagttttgggcttcaagagtcagccacgaggcaggcactaggcctggaaatggcctcacagtcatgagttgggcctaaatgggccactgtgagggaggagctgtgcctgttgaggctgctggcaggcaggcagaaatttggcctggggcagctgccatgaggcaagagctgggcctggaaaaagcccctgggaggcaagagcagggcctgcagaggctgttctcaagtcaaagctgggcctgttgatgccaccgggaagcagaaggtgggcctggagagtttgacttgaggaagttttgggcctacattggccgccatgagctggacaggaactgggccaaaaaaggctgttgtgaggcagcagttgtgcctgtagacccagc

SEQ ID NO: 87 - DNA, 495 bp, Homo sapiens
  source 1..495: mol_type = genomic DNA; note = chr16:90166977-90167471
cctgaggatgccacagtgagacaccatctgagtctggagggtccactgtgaggcagaggctggcctgtagagtccaacagtagacagaagttgggcaaaaggctgatttgaggaagttttgggcttcaagagtcagccacgaggcaggcactaggcctggaaatggcctcacagtcatgagttgggcctaaatgggccactgtgagggaggagctgtgcctgttgaggctgctggcaggcaggcagaaatttggcctgggcagctgccacgaggcaagagctgggcctggaaaaagcccctgggaggcaagagcagggcctgcagaggctgttctcaagtcaaagctgggcctgttgatgccaccgggaagcagaaggtgggcctggagagtttgacttgaggaagttttgggcctacattggccgccatgagctggacaggaactgggccaaaaaaggctgttgtgaggcagcagttgtgcctgtagacccagc

SEQ ID NO: 88 - DNA, 496 bp, Homo sapiens
  source 1..496: mol_type = genomic DNA; note = chr1:227968386-227968881
cctgaggatgccacagtgagacaccatctgggtctggagggtccactgtgaggcagaggctggcctgtagagtccgacagtagacagaagttgggcaaaaggctgatttgaggaagttttgggcttcaagagtcagccacgaggcaggcactaggcctggaaatggcccgacagtcatgagttgggcctaaatgggccactgtgagggaggagctgtgcctgttgaggctgctggcaggcaggcagaaatttggcctggggcagctgccatgaggcaggagctgggtctggaaaaagcccctgggaggcaagagcagggcctgcagaggctgttctcaagtcaaagctgggcctgttcatgccaccgggaagcagaaggtgggcctggagagtttgacttgaggaagttttgggcctacattggccgccatgagctggacaggaactgggccaaaaaaggctgttgtgaggcagcagttgtgcctgtagacccagc

SEQ ID NO: 89 - DNA, 499 bp, Homo sapiens
  source 1..499: mol_type = genomic DNA; note = chr4:118631285-118631783
cctgaggatgccacagtgagacaccatctgggtctggagggtccactgtgaggcagaggctggcctgtagagtccgacagtagacagaagttaggcaaaaggctgatttgaggaagttttgggcttcaagaaagagtcagccaggaggcaggcactaggcctggaaatggcccgacagtcatgagtggggcctaaatgggccactgtgagggaggagctgtgcctgttgaggctgctggcaggcaggaagaaatttggcctggggcagctgccatgaggcaagagctgggcttggaaaagcccctgggaggcaagagcagggcctgcagaggctgttctcaagtcaaagctgggcctgttcatgccaccaggaagcagaaggtgggcctggagagtatgacctgaggaagttttgggcctacattggccgccatgaactggacaggaactgggccaaaaaaggctgttgtgaggcagcagttgtgcctgtagactcagc

SEQ ID NO: 90 - DNA, 496 bp, Homo sapiens; identical to SEQ ID NO: 74
  source 1..496: mol_type = genomic DNA; note = chr5:181324898-181325393

SEQ ID NO: 91 - DNA, 496 bp, Homo sapiens
  source 1..496: mol_type = genomic DNA; note = chr7:128652019-128652514
actgaggatgccacagtgagacaccatctgggtctggagggtccactgtgaggcagaggctggcctgtagagtccgacagtagacagaagttgggcaaaaggctgatttgaggaagttttgggcttcaagagtcagccacgaggcaggcactaggcctggaaatggcccgacagtcatgagttgggcctaaatgggccactgtgagggaggagctatgcctgttgaggctgctggcaggcaggcagaaatttggcctggggcagctgccatgaggcaagagctgggcctggaaaaagcccctgggaggcaagagcagggcctgcagaggctgttctcaagtcaaagctgggcctgttcatgccactgggaagcagaaggtgggcctggagagtttgacttgaggaagttttgggcctacattggccgccatgagctggacaggaactgggccaaaaaaggctgttgtgaggcagcagttgtgcctgtagacccagc

SEQ ID NO: 92 - DNA, 509 bp, Homo sapiens
  source 1..509: mol_type = genomic DNA; note = chr7:63746246-63746754
cctgaggatgccacagtgagacaccatctgggtctggagggtccactgtgaggcagaggctggcctgtagagtccgacagtagacagaagttgagcaaaaggctaatttcaggaagttttgggcttcaagagtcagccaggaggcaggcactaggcctggaaatggcccgacagtcatgagttgggcctaaatgggccactgtgagggaggagctgtgcctgttgaggctgctggcaggcaggcagaaacttggcctggagcagcttccatgagacaagagctgggcctggagaagcccctgagaggtaagaggagggcctgcagaggctgttctcaagtcaaagctgggcctgttcatgccaccgggaagcagaaggtgggcctggagagttttacttgaggaagttttgggcctacatttgctgccgtgagctggacaggaactgggccaaaaaaggctgttgtgaggcagctgttgtgaggcagcagttgtgcctgtagactcagc

SEQ ID NO: 93 - DNA, 492 bp, Homo sapiens
  source 1..492: mol_type = genomic DNA; note = chr7:45808335-45808826
cctgaggatgccacagtgagacaccatctgggtctggagggtccactgtgaggcagaggctggcctgtagagtccgacagtagacagaagttgggcaaaaggctgatttgaggaagttttgggcttcaagagtcagccaggaggcaggcactaggcctggaaatggcccgacagtcatgagttgggcctaaatgggccactgtgagggaagagctgtgcctgttgaggctgctggcaggcaggcagaaacttggcctggggcagccgccatgaggcaagagctgggcctggagaagcccctgggaggcaagagcagggcctgcagaggctgttctcaagtcaaagctgggcctgttcatgccaccaggaagaaggcaggcctggagagtttgacttgaggaagttttgggcctacattggtcgctgtgagctggacaggaactgggccaaaaaaggctgttgtgaggcagcagttgtgcctgtagactcagc

SEQ ID NO: 94 - DNA, 24 bp, synthetic construct
  source 1..24: mol_type = other DNA; note = IC primer 1
gtagctccataatcagaagatgcg

SEQ ID NO: 95 - DNA, 27 bp, synthetic construct
  source 1..27: mol_type = other DNA; note = IC primer 2
attcgctctactccaaccacataatag

SEQ ID NO: 96 - DNA, 434 bp, Homo sapiens
  source 1..434: mol_type = genomic DNA; note = IC template
gtagctccataatcagaagatgcgctaccgtacacaacggcggtttaaactttgtagacctcacgcggctgctcaggtcttgcgcatcccatacttcacatggacggaaagctgtgggtttgtatcgataagccgtaggttcctaggatcgagtcacccaaacaacttcgagagctcttctcaccaaccctttgaactccgaaatgccgtgaacacatccccttagttggcattggtctactaaccgagtatcgtacaaagcgagacaggtctggcccctactgcttggatgcgaactaacagtgttcctatatccgtttatactgtggtcgatttcgaaccgatatctcaaagagagcgccggcagagccttagtatcggaactggcgagtgtgtttgggtgaggactattatgtggttggagtagagcgaat

SEQ ID NO: 97 - DNA, 26 bp, synthetic construct
  source 1..26: mol_type = other DNA; note = IC probe
tccgatactaaggctctgccggcgct

(19) United States
(12) Patent Application Publication
(10) Pub. No.: US 2026/0146282 A1
(43) Pub. Date: May 28, 2026

(54) QUANTIFICATION OF NUCLEIC ACIDS AND/OR ASSESSMENT OF THE STATUS OF NUCLEIC ACID DEGRADATION AND/OR INTEGRITY BY DIGITAL PCR

(71) Applicant: QIAGEN GmbH, Hilden (DE)

(72) Inventors: Miroslav VRANEŠ, Hilden (DE); Stefan Otto CORNELIUS, Hilden (DE); Ralf PEIST, Hilden (DE)

(21) Appl. No.: 19/123,692

(22) PCT Filed: Oct. 24, 2023
(86) PCT No.: PCT/EP2023/079628

§ 371 Date: Apr. 23, 2025

(30) Foreign Application Priority Data
Oct. 25, 2022 (EP) 22203693.1

(51) Int. Cl. C12Q 1/6851

(52) U.S. Cl. CPC C12Q 1/6851

(57) ABSTRACT

The present invention relates to a method for assessing the status of nucleic acid degradation and/or integrity of one or more nucleic acids in a sample, comprising the steps of digitally amplifying at least two regions within at least one locus, and detecting the amount of the at least two amplification products through the use of at least two probes. In the present invention preferably a nanoplate based dPCR method is applied.

### FIELD OF INVENTION

The present invention is in the field of molecular biology, diagnostics, and more particular, in the field of analytical and forensic sciences. Furthermore, the invention is in the field of nucleic acid amplification and quantification, more specifically in the field of DNA quantification and assessment of DNA degradation and integrity.

### BACKGROUND

DNA based diagnostic methods are of rising importance in many areas, for example in the fields of diagnostic-, genetic-, forensic-, and food testing or for the detection of genetically modified organisms (GMO).

The determination of the quantity of DNA recovered from forensic samples as well as from other samples is a critical step in the overall DNA typing process, but also in the detection of DNA in various other fields of science. A narrow range of input DNA from 0.5 to 2 ng is often needed to produce optimal results with for example multiplex DNA typing kits. Therefore, in order to ensure that a positive result is a positive result and/or a negative result is a negative result due to the absence of DNA, quantification of DNA is of absolute importance. Furthermore, the quality of standards for forensic DNA testing laboratories requires human-specific DNA quantification. This is due to isolation techniques that can recover human DNA as well as bacterial and other exogenous DNA.

In addition, the quality of the DNA isolated from various sources can be compromised by degradation. This can influence the precision and validity of the assays applied on the tested DNA. In forensics for example, human DNA integrity is of great importance and the quantification of the DNA is often put in front of a DNA fingerprinting analysis, e.g. in front of STR analyses or next-generation-sequencing (NGS). Currently, quantitative real-time PCR (qPCR) is a widely used method for DNA quantification.

As reviewed in Quan et al. (Quan et al., 2018, MDPI, “dPCR: A Technology Review”), quantitative real-time PCR is based on conventional PCR. In conventional PCR, target DNA is amplified in multiple cycles, wherein at the end of each of a 100% efficient PCR cycle the number of target DNA molecules is doubled (exponential amplification). Hence, 2ⁿ copies can theoretically be produced after n cycles. However, in practice, the PCR reagents will be depleted at one point and amplification products will self-anneal, resulting in reduced amplification efficiency until a plateau is reached and the amplification process saturates. At the end of a PCR reaction, the amplified products can be analyzed using agarose gel electrophoresis (end-point measurement). The specificity of a conventional PCR relies on sequence hybridization. The sensitivity of a conventional PCR depends on enzyme-based amplification (Quan et al., 2018, MDPI, “dPCR: A Technology Review”).

As mentioned above, quantitative real-time PCR is based on conventional PCR, but the amount of amplified PCR products are measured after each amplification cycle using a fluorescent readout. A typical real-time PCR amplification plot shows a sigmoidal-shaped curve (on a linear scale) and includes a baseline phase, followed by an exponential phase that reaches a plateau via a linear phase. The most efficient phase of amplification is represented by the exponential phase and the amount of amplified PCR products doubles with each cycle (at an amplification efficiency of 100%). A relative quantification of a target to a calibrator is enabled by real-time PCR. When calibrated with a standard curve using data from the exponential phase, the method is quantitative (qPCR). The ‘absolute’ amount of target sequence in a qPCR reaction is measured relative to a standard curve, which is generated from a sample of known quantity or copy number. This method implies that the amplification efficiencies of the standards and the sample are equivalent. Differences in PCR efficiencies can significantly affect the accuracy of the quantification (Quan et al., 2018, MDPI, “dPCR: A Technology Review”).

qPCR is a widely used method for DNA quantification since it offers various advantages. Such advantages are, within others, the species specific identification, a wide dynamic range as well as the ease of automation. For a typical short tandem repeats analysis markers range from about 100 bp up to about 450 bp, depending on the loci analyzed. Degraded DNA can lead to loss of amplification of the longest systems, or even to a complete failure of STR analysis.

As noted before, another important parameter in forensics and other disciplines is the degradation grade of the DNA that is analyzed. The extend of degradation is determined by the comparison of the amount of a short amplified DNA fragment with the amount of a long amplified DNA fragment obtaining a DNA degradation ratio (Goecker et al. 2016, Forensic Science International: “Comparison of Quantifiler® Trio and InnoQuant™ human DNA quantification kits for detection of DNA degradation in developed and aged fingerprints”). Furthermore, it is known from literature that the use of a short DNA fragment and one long DNA fragment exhibit a positive relationship between an increasing DNA degradation ratio and a loss in longer STR alleles. According to Goecker et al 2016, different quantification kits available utilize different targets for the DNA degradation assessment. When applied to the same degraded DNA sample, the use of different DNA targets in different qPCR systems may lead to significant different results for the sensitivity of DNA quantitation and the extent of DNA degradation.

For the DNA quantification by qPCR a standard curve is needed to calculate DNA amount of the sample. Usually the standard curve is prepared by a serial dilution by the user. The preparation of the standard curve is critical for a precise DNA quantification of samples. Depending on the user's skills and quality of the used equipment for standard curve preparation the process of DNA dilution and preparation of the DNA standard curve itself can be very tedious, laborious and error prone which can lead to wrong quantification results and a complete failure of STR analysis.

Furthermore, methods using digital PCR to quantify human DNA have been limited due to the availability of different dye channels making it impossible to higher multiplex methods to assess DNA quantity and DNA integrity at the same time.

A sensitive DNA quantification method to accurately detect and quantify DNA and in parallel to assess DNA degradation or integrity for a wide range of disciplines is therefore of great interest and there is a need for a method to encounter the drawbacks of the currently available kits.

The present invention solves the above identified problem and provides for the following solution as outlined below.

### SUMMARY OF THE INVENTION

The present invention provides for a highly sensitive method for the assessment of the status of nucleic acid degradation and/or integrity of one or more nucleic acids in a sample, comprising the steps of amplifying at least two genomic regions within at least one genomic locus, wherein the amplification is done by digital PCR, and detecting the amount of the at least two amplification products through the use of at least two probes.

Preferably, at least one of the at least two probes binds to one of the at least two amplification products and at least one other probe of the at least two probes binds to another one of the at least two amplification products.

Specifically, in one embodiment the present invention relates to the provision of a highly sensitive method for the assessment of the status of nucleic acid degradation and/or integrity of one or more nucleic acids in a sample, comprising the steps of amplifying at least two overlapping regions within at least one genomic locus, and detecting the amount of the at least two amplification products through the use of at least two probes, wherein at least one probe binds to the region of overlap and at least one other probe binds to a non-overlapping region, wherein the amplification method is Digital Polymerase Chain Reaction (dPCR).

In an alternative embodiment of the present invention, the herein described method provides for the assessment of the status of nucleic acid degradation and/or integrity of one or more nucleic acids in a sample, comprising the steps of amplifying at least two non-overlapping genomic regions within at least one genomic locus, and detecting the amount of the at least two amplification products through the use of at least two probes, wherein the amplification method is Digital Polymerase Chain Reaction (dPCR).

Preferably, at least one of the at least two probes of the above embodiment of the invention binds to one of the at least two amplification products and at least one other probe of the at least two probes binds to another one of the at least two amplification products.

In yet another alternative embodiment of the present invention, the herein described method provides for the assessment of the status of nucleic acid degradation and/or integrity of one or more nucleic acids in a sample, comprising the steps of amplifying at least two non-overlapping genomic regions within at least two genomic loci, and detecting the amount of the at least two amplification products through the use of at least two probes, wherein the amplification method is Digital Polymerase Chain Reaction (dPCR).

Preferably, at least one of the at least two probes of the above embodiment of the invention binds to one of the at least two amplification products and at least one other probe of the at least two probes binds to another one of the at least two amplification products.

According to several embodiments of the invention, the at least two genomic regions may be overlapping and are amplified using at least one common primer.

According to several embodiments of the invention, the at least one of the at least two probes binds to the region of overlap of said at least two amplification products and at least one other probe binds to a non-overlapping region of said at least two amplification products.

According to several embodiments of the invention, the size of the amplification products of the at least two genomic regions is between 20 base pairs and 2000 base pairs long.

According to several embodiments of the invention, wherein if two genomic regions are amplified, the second amplification product is at least 30% longer than the first amplification product.

According to several embodiments of the invention, if a third genomic region is amplified, the third amplification product is at least 30% longer than the second amplification product.

According to several embodiments of the invention, the at least one genomic locus is a single copy locus (SCL) or a multicopy locus (MCL) within the nucleic acid assessed for integrity and/or degradation.

According to several embodiments of the invention, the method obtains a degradation index of at least 30 when measuring degraded DNA of 200 bp length and of at least 150 when measuring degraded DNA of 150 bp length.

According to several embodiments of the invention, in parallel to the assessment of the status of DNA degradation and/or integrity of one or more nucleic acids in a sample, the one or more nucleic acids are quantified and/or detected.

According to several embodiments of the invention, the sample is selected from the group of genomic samples comprising, but not limited to, human-, animal-, plant-, bacterial-, archaeal-, oomycetric, autosomal, human-autosomal or fungal DNA, environmental samples and food samples.

According to several embodiments of the invention, the dPCR method is a nanoplate based dPCR method.

According to several embodiments of the invention, the dPCR is at least a triplex dPCR, and wherein the amplified genomic regions comprise a human target, a degradation target and an internal amplification control region.

The invention further relates to a kit comprising at least one of the primers and/or probes corresponding to SEQ ID NOs 1-8 or complements thereof, and optionally SEQ ID NOs 94-95 and 97, or complements thereof, for assessing the status of nucleic acid degradation and/or integrity of one or more nucleic acids using digital PCR amplification. SEQ ID NO 96 may also optionally be included into the kit.

Digital polymerase chain reaction (dPCR; also abbreviated as e.g. digital PCR, DigitalPCR, ddPCR or dePCR) is a refinement of conventional polymerase chain reaction methods such as qPCR. Digital polymerase chain reaction (dPCR) enables the absolute quantification of target nucleic acids present in a sample and alleviates the shortcomings of qPCR. Unlike qPCR, dPCR does not rely on calibration curves for sample quantification. Hence, it avoids the pitfalls associated with variations in reaction efficiencies. dPCR is a method of absolute nucleic acid quantification that hinges on the detection of end-point fluorescent signals and the enumeration of binomial events, i.e. absence or presence of fluorescence in a partition. In dPCR, the sample is first partitioned into many independent PCR sub-reactions such that each partition contains either a few, one or no target sequences. Such partitions or microreactors can be arranged for example, but not limited to, by small water-in-oil droplets or by microfluidic nanoplates.

After PCR, the fraction of amplification-positive partitions is used to quantify the concentration of the target sequence with a statistically defined accuracy using Poisson's statistics, wherein partitions with either 0, 1, or more target sequences are each needed for the calculations. Interestingly, sample partitioning efficiently concentrates the target sequences within the isolated microreactors. This concentration effect reduces template competition and thus enables the detection of rare mutations in a background of wild-type sequences (Quan et al., 2018, MDPI, “dPCR: A Technology Review”). dPCR may also allow for a higher tolerance to inhibitors present in a sample, because there is no need to have an amplification efficiency per cycle of almost 100%, as required for qPCR. Instead, it is sufficient if at the end of the amplification reaction either a signal or no signal is detectable. PCR carries out one reaction per single sample. dPCR also carries out a single reaction within a sample, however the sample is separated into a large number of partitions and the reaction is carried out in each partition individually. This separation allows a more reliable collection and sensitive measurement of nucleic acid amounts.

Instead of performing one reaction per well, dPCR involves partitioning the PCR solution into at least a few hundred, but in most cases several thousand or tens of thousands or more of nano-liter sized partitions, where a separate PCR reaction takes place in each one. A dPCR solution is made similarly to a quantitative assay either using fluorescence-quencher probes or intercalating dyes, and a PCR master mix, which contains DNA polymerase, dNTPs, MgCl2, and reaction buffers at optimal concentrations.

Several different methods can be used for sample partitioning, including microwell plates, microfluidic nanoplates, capillaries, oil emulsion, and arrays of miniaturized chambers with nucleic acid binding surfaces.

After multiple PCR amplification cycles, the samples are checked for fluorescence with a binary readout of “0” (absence) or “1” (presence). The fraction of fluorescing partitions is recorded. The partitioning of the sample allows one to estimate the number of different molecules by assuming that the molecule population follows the Poisson distribution, thus accounting for the possibility of multiple target molecules inhabiting a single partition. Using Poisson's law of small numbers, the distribution of target molecule within the sample can be accurately approximated allowing for a quantification of the target strand in the PCR product.

In contrast to a qPCR reaction, a dPCR reaction is an endpoint PCR reaction. dPCR uses the number of fluorescence-positive partitions over the total to back-calculate the target concentration. In contrast to qPCR, calibration curves are not needed for sample quantification in dPCR. All in all, compared to qPCR, dPCR provides a more robust quantification, is less prone to inhibitors and is independent of a quantification standard.

The benefits of dPCR include increased precision through massive sample partitioning, which ensures reliable measurements in the desired DNA sequence due to reproducibility. Error rates are larger when detecting small-fold change differences with qPCR, while error rates are smaller with dPCR due to the smaller-fold change differences that can be detected in DNA sequence. Also, dPCR is highly quantitative as it does not rely on relative fluorescence of the solution to determine the amount of amplified target DNA.

The inventors have now astonishingly found that applying dPCR to a previously published method published in WO2018050844 results in entirely unexpectedly good results. The new method is dramatically more precise than the known method.

The invention further relates to a method of designing primers and/or probes for amplifying at least two genomic regions within at least one genomic locus, wherein the locus is a single copy locus (SCL) or multicopy locus (MCL).

The invention further relates to a method of designing primers and/or probes for amplifying at least two overlapping genomic regions within at least one genomic locus, wherein the locus is a single copy locus (SCL) or multicopy locus (MCL).

The invention further relates to a method of designing primers and/or probes for amplifying at least two non-overlapping genomic regions within one genomic locus, wherein the locus is a single copy locus (SCL) or multicopy locus (MCL).

The invention further relates to a method of designing primers and/or probes for amplifying at least two non-overlapping genomic regions within at least two genomic loci, wherein both loci are either single copy loci (SCL) or multicopy loci (MCL).

The invention also relates to primer and primer pairs for amplifying said overlapping regions within at least one locus.

The invention also relates to primer and primer pairs for amplifying said non-overlapping regions within at least one locus.

The primers and probes according to the invention may be in a kit, the kit further optionally including the primers and probe for the internal amplification control. Hence, the invention also relates to a kit for assessing the status of nucleic acid degradation and/or integrity of one or more nucleic acids, and optionally to report successful digital PCR amplification in a sample. In particular, the invention relates to a kit comprising the primers and probes corresponding to SEQ ID NOs 1-8, and optionally SEQ ID NOs 94-95 and 97, or complements thereof, for assessing the status of nucleic acid degradation and/or integrity of one or more nucleic acids, and optionally to report successful digital PCR amplification in a sample. SEQ ID NO 96 may also optionally be included into the kit. The kit optionally further comprises one or more reagents needed to perform the methods of the invention, e.g. one or more intercalating dyes, a DNA polymerase, dNTPs, MgCl2, and a reaction buffer.

In one embodiment of the invention, the dPCR is at least a triplex dPCR, wherein the amplified genomic regions comprise a human target, a degradation target and an internal amplification control region. Such a triplex dPCR enables simultaneous quantification of human DNA, assessment of DNA integrity and report of successful or unsuccessful amplification. Details are described and shown in FIGS. 4 and 6.

### DETAILED DESCRIPTION OF THE INVENTION

As outlined above, the assessment of the integrity and/or degradation status of a nucleic acid template is of great importance and currently available methods do not sufficiently address and/or solve this problem. The present invention, however, solves the problem and provides for a valuable system to assess the status of DNA degradation and/or integrity which is highly superior over existing methods and shows an astonishing sensitivity that has not been expected before.

The herein described method of the present invention provides for the assessment of the status of nucleic acid degradation and/or integrity of one or more nucleic acids in a sample, comprising the steps of amplifying at least two genomic regions within at least one genomic locus, wherein the amplification is done by digital PCR, and detecting the amount of the at least two amplification products through the use of at least two probes, wherein at least one of the at least two probes binds to one of the at least two amplification products and at least one other probe of the at least two probes binds to another one of the at least two amplification products.

The terms “genomic regions” or “regions” as used herein can be used interchangeably within the context of genomic regions that are amplified, and can either refer to overlapping or non-overlapping regions, if it is not clearly indicated to which one of them the term refers to. The same applies to their singular forms.

The terms “genomic locus” or “locus” are interchangeable. The same applies to their plural forms.

The term “at least one” as used in the present invention refers to 1, 2, 3, or more. For example, at least one region or at least one locus is meant to include 1, 2, 3, or more regions or loci, respectively. Similarly, “at least two” as used herein refers to 2, 3, 4 or more. For example, at least two regions or at least two loci is meant to include 2, 3, 4 or more regions or loci, respectively.

In a first aspect of the present invention, the herein described method provides for the assessment of the status of nucleic acid degradation and/or integrity of one or more nucleic acids in a sample, comprising the steps of amplifying at least two overlapping regions within at least one genomic locus, and detecting the amount of the at least two amplification products through the use of at least two probes, wherein at least one probe binds to the region of overlap and at least one other probe binds to a non-overlapping region.

One novel key feature of the first aspect of the present invention is that the at least two overlapping regions are amplified by using at least one common primer and the method for amplification is a digital PCR method.

In a second aspect of the present invention, the herein described method provides for the assessment of the status of nucleic acid degradation and/or integrity of one or more nucleic acids in a sample, comprising the steps of amplifying at least two non-overlapping genomic regions within at least one genomic locus, and detecting the amount of the at least two amplification products through the use of at least two probes, wherein at least one of the at least two probes binds to one of the at least two amplification products and at least one other probe of the at least two probes binds to another one of the at least two amplification products. The method for amplification is a digital PCR method.

In a third aspect of the present invention, the herein described method provides for the assessment of the status of nucleic acid degradation and/or integrity of one or more nucleic acids in a sample, comprising the steps of amplifying at least two non-overlapping genomic regions within at least two genomic loci, and detecting the amount of the at least two amplification products through the use of at least two probes, wherein at least one of the at least two probes binds to one of the at least two amplification products and at least one other probe of the at least two probes binds to another one of the at least two amplification products. The method for amplification is a digital PCR method.

According to several embodiments of the invention, the at least two genomic regions may be overlapping and are amplified using at least one common primer.

According to several embodiments of the invention, the at least one of the at least two probes binds to the region of overlap of said at least two amplification products and at least one other probe binds to a non-overlapping region of said at least two amplification products.

According to several embodiments of the invention, the size of the amplification products of the at least two genomic regions is between 20 base pairs and 2000 base pairs long.

According to several embodiments of the invention, wherein if two genomic regions are amplified, the second amplification product is at least 30% longer than the first amplification product.

According to several embodiments of the invention, if a third genomic region is amplified, the third amplification product is at least 30% longer than the second amplification product.

The second amplification product may be at least 10%, 15%, 20%, 25%, 30%, 35%, 40%, 45%, or 50% longer than the first amplification product. If three amplification products are present, the third one may be at least 10%, 15%, 20%, 25%, 30%, 35%, 40%, 45%, or 50% longer than the second amplification product. If four amplification products are present, the fourth one may be at least 10%, 15%, 20%, 25%, 30%, 35%, 40%, 45%, or 50% longer than the third amplification product, and so on. Hence, preferably, the amplification products are each of different sizes. Alternatively, only one of the at least two amplification products has a different size, which may be at least 10%, 15%, 20%, 25%, 30%, 35%, 40%, 45%, or 50% longer than at least one other amplification product.

According to several embodiments of the invention, the at least one genomic locus is a single copy locus (SCL) or a multicopy locus (MCL) within the nucleic acid assessed for integrity and/or degradation.

According to several embodiments of the invention, the method obtains a degradation index of at least 30 when measuring degraded DNA of 200 bp length and of at least 150 when measuring degraded DNA of 150 bp length.

According to several embodiments of the invention, in parallel to the assessment of the status of DNA degradation and/or integrity of one or more nucleic acids in a sample, the one or more nucleic acids are quantified and/or detected.

According to several embodiments of the invention, the sample is selected from the group of genomic samples comprising, but not limited to, human-, animal-, plant-, bacterial-, archaeal-, oomycetric, autosomal, human-autosomal or fungal DNA, environmental samples and food samples.

According to several embodiments of the invention, the dPCR method is a nanoplate based dPCR method.

According to several embodiments of the invention, the dPCR is at least a triplex dPCR, and wherein the amplified genomic regions comprise a human target, a degradation target and an internal amplification control region.

The invention further relates to a kit comprising at least one of the primers and/or probes corresponding to SEQ ID NOs 1-8 or complements thereof, and optionally SEQ ID NOs 94-95 and 97, or complements thereof, for assessing the status of nucleic acid degradation and/or integrity of one or more nucleic acids using digital PCR amplification. SEQ ID NO 96 may also optionally be included into the kit.

Herein, numerous different kinds of digital PCR may be used.

### Droplet Digital PCR

Droplet Digital PCR (ddPCR) is a method of dPCR in which a for example a 20 microliter sample reaction including assay primers and either Taqman probes or an intercalating dye, is divided into about 20,000 nanoliter-sized oil droplets through a water-oil emulsion technique, thermocycled to endpoint in a 96-well PCR plate, and fluorescence amplitude read for all droplets in each sample well in a droplet flow cytometer.

### Chip-Based Digital PCR

Chip-based Digital PCR (dPCR) is also a method of dPCR in which the reaction mix (also when used in qPCR) is divided into at least a few hundred, but in most cases several thousand, or about 10,000 to about 45,000, or tens of thousands, or hundreds of thousand or more of partitions on a chip, then amplified using an endpoint PCR thermocycling machine, and is read using a high-powered camera reader with fluorescence filter for all partitions on each chip. Several fluorescent dyes may be used, as long as the corresponding fluorescent channels can be separated from each other. Examples for usable dyes are HEX, FAM, Cy5, Cy5.5 and Texas Red. Such a chip-based method relies on a nanofluidic chip.

QIAcuity plate based dPCR (QIAGEN) is preferred. This method is based on partitions in a plate for example and not oil drops. Here, ideally each partition that contains a target DNA molecule creates a signal, giving rise to no signal in partitions where no target DNA is, hence the word digital, yes/no.

The inventors surprisingly found that particularly good results are achieved when, the size of the dPCR amplification products of the at least two regions are between 20 base pairs and 2000 base pairs long for this digital PCR method. Ideally, the amplification product of one region is between 60 base pairs and 200 base pairs long, more ideally between 80 and 150, and most ideally between 85 and 100 base pairs long. In one ideal embodiment, one of the amplification products is 91 base pairs long.

It is also preferred that the amplification product of a second region is between 200 and 2000 base pairs (bp) long, more preferred between 210 and 1500 bp, between 220 and 1000 bp, between 230 and 800 bp, between 240 and 500 bp, or between 250 and 400 bp. Most preferably, the amplification product of a second region is 353 base pairs long.

Astonishingly it has turned out that, if the amplification product of a second region is longer than the amplification product of a first region better results may be achieved.

Therefore, in another preferred embodiment according to the invention, at least one of the amplification products of the at least two regions is at least 30% longer than another one.

If a third region is amplified, it is preferred that the amplification product generated is at least 30% longer than the second amplification product.

It was very surprising for the inventors that with the system and arrangement according to the invention, the degraded DNA can be detected with such a particular high sensitivity.

In one aspect of the present invention, the locus is a single copy locus (SCL) or a multicopy locus (MCL) within the nucleic acid assessed for integrity and/or degradation.

In case the locus is a multicopy locus, it may be found on various chromosomes and may be present many times in the genome. This can further improve the detection sensitivity of the present method and may provide a further valuable advantage of the present method. In case the locus is a single copy locus, it may be present only once in the genome.

In another aspect according to the present invention, it is particularly useful that in parallel to the assessment of the status of DNA degradation and/or integrity of one or more nucleic acids in a sample, the one or more nucleic acids are quantified and/or detected.

The degradation status/integrity of DNA can be assessed by using for example, at least two differently sized genomic regions in a dPCR assay. Preferably, the amplified targets either have comparable copy numbers, or the lack of it is accounted for in subsequent calculations. In case of degraded DNA the mean length of the DNA fragments in the sample will decrease leading to less amplifiable templates of the longer PCR systems. As a result, the amount of probes binding to the longer amplification products decreases and the ratio of the measured quantity of a smaller amplicon over the quantity of a larger amplicon (degradation index) increases. Hence, the higher the degradation index, the lower the integrity or the higher the degradation status of the DNA.

Therefore, in one aspect of the present invention, the status of DNA integrity and/or degradation is expressed by the ratio of the quantification of the at least two regions within the at least one locus.

As shown in FIG. 3, the method according to the invention shows the benefits of the present method in a degradation indices. It was very astonishing for the inventors to find out that the present method shows a much higher sensitivity than existing systems, in particular, when measuring degraded DNA of small sized fragments.

As such, the method according to the invention also relates to a method wherein the degradation index is at least 2 times higher for degraded fragments of 200 bp length.

Ideally, the method according to the invention obtains a degradation index of at least 30 when measuring degraded DNA of 200 bp length and of at least 150 when measuring degraded DNA of 150 bp length. As shown in FIG. 2, the difference between 300 bp vs 200 bp vs 150 bp is relatively small for system 1. This means that degraded DNA with an average size of 280 bp or 240 bp is more difficult to distinguish from 300 bp. System 2 can better discriminate fragments of 300 bp, 200 bp and 150 bp length, because the delta in the DI between the small fragment sizes is larger and can be better used to resolve the range and intermediate fragment sizes.

The degradation index can sensibly vary according to the concentration of DNA fragment in a degraded DNA sample.

The applicant developed a method for quantifying and assessing DNA integrity/degradation with an up to 70× higher precision than state of the art methods. Not only the degradation index (i.e. DNA intact or degraded) is important for the success of DNA profiling, e.g. via STRs, but also the precise measurement of the quantity of DNA in a sample. The more accurate the determination of the amount of amplifiable DNA the more successful the DNA profiling. FIG. 1 shows that the precision and reliability of DNA quantification using a method of the invention (dPCR) outperforms other state of the art qPCR methods as can be seen in the low % CV (coefficient of variation) values. In addition to an improved DNA quantification, measurement of DNA integrity/degradation status is also improved by a method of the present invention. FIG. 3 clearly demonstrates that the method according to the invention consistently obtains much higher indices than other state of the art techniques, in particular for the smaller fragments ranging from 300 bp to 150 bp in size. This indicates a much higher sensitivity/precision for the detection of degraded DNA and better resolution of DNA integrity.

Further specifics of the good results obtained in a method according to the invention are detailed in the Figure legends and Examples.

In different embodiments that are all equally preferred, the sample is selected from the group of genomic samples, such as human DNA or microbial DNA (e.g. bacterial, archaeal or fungal), food samples (e.g. animal or plant derived), environmental samples (e.g. containing microorganisms). The method according to the invention can also be used for human/animal pathogen testing (bacterial, fungal, oomycetes) and phytopathology (bacterial, fungal, oomycetes), using corresponding samples.

In different embodiments that are all equally preferred, the sample is selected from the group of genomic samples comprising, but not limited to, human-, animal-, plant-, bacterial-, archaeal-, oomycetric, autosomal, human-autosomal or fungal DNA, environmental samples and food samples.

According to another embodiment, the sample subjected to the present method originates from one of the following specimens comprising whole blood, blood fractions, oral fluids, body fluids, human bioptic tissue or other parts of the human body upon availability for isolation of a genome. As used herein the terms “oral fluids” and “body fluids” refers to fluids that are excreted or secreted from the buccal cavity and from the body, respectively, from which a genome can be isolated. As a non-limiting example, oral and body fluids may comprise saliva, sputum, swab, urine.

In case of a forensic sample, the sample may comprise a mixture of male and female DNA wherein the amount of female DNA exceeds the amount of male DNA by several orders of magnitude, e.g. in sexual assault samples or in blood samples of pregnant women comprising male fetal DNA. Thus, according to another embodiment, the sample comprises one or more additional nucleic acids originating from a different genome. As used herein, the term “different genome” refers to genome isolated from a different subject, generally identified as female DNA.

In different embodiments, the dPCR method is a nanoplate based dPCR method.

The dPCR amplification methods will comprise buffers, dNTPs or NTPs in addition to the enzymes required.

As used herein, the term “dNTP” refers to deoxyribonucleoside triphosphates. Non-limiting examples of such dNTPs are dATP, dGTP, dCTP, dTTP, dUTP, which may also be present in the form of labelled derivatives, for instance comprising a fluorescent label, a radioactive label, a biotin label. dNTPs with modified nucleotide bases are also encompassed, wherein the nucleotide bases are for example hypoxanthine, xanthine, 7-methylguanine, inosine, xanthinosine, 7-methylguanosine, 5,6-dihydrouracil, 5-methylcytosine, pseudouridine, dihydrouridine, 5-methylcytidine. Furthermore, ddNTPs of the above-described molecules are encompassed in the present invention.

As used herein, the term “NTP” refers to ribonucleoside triphosphates. Non-limiting examples of such NTPs are ATP, GTP, CTP, TTP, UTP, which may also be present in the form of labelled derivatives, for instance comprising a fluorescent label, a radioactive label, a biotin label.

According to another embodiment of the present invention, the amplification reaction comprises, (a) Tris-HCl at a pH of between 8 and 8.8 (at 20° C.) and/or, (b) potassium salt selected from the group of, potassium chloride and potassium sulphate and/or, (c) an ammonium salt, preferably ammonium chloride or ammonium sulphate and/or, (d) magnesium chloride and/or, (e) a hot-start polymerase.

Preferably, the concentration of Tris-HCl is in the range from 10 to 100 mM, most preferably in the range from 20 to 70 mM, the concentration of K⁺ is in the range from 1-25 mM, most preferred in the range from 2.5 to 20 mM, the concentration of NH₄⁺ in range from 1 to 40 mM, most preferred in the range from 2.5 to 30 mM, and a concentration of Mg²⁺ of 0.5 mM to 8 mM in excess to the concentration of the four dNTP's, most preferred a concentration of Mg²⁺ of 0.7 mM to 5 mM in excess to the concentration of the four dNTP's, a hot-start polymerase, preferentially a hot-start polymerase allowing a hot-start time of less than 5 min, most preferred below 2 min.

Overall, the method according to the invention shows an improved sensibility over prior art and other commercially available methods. In particular, the present method provides for a short run time, good accuracy, and low sensitivity to inhibitors.

### EXAMPLES

The following example are used in conjunction with the Figures and Tables to illustrate the invention.

Herein,
dPCR refers to digital PCR;
qPCR refers to real-time quantitative polymerase chain reaction;
QPro refers to QIAGEN Investigator Quantiplex Pro Kit;
QHYres refers to QIAGEN Investigator Quantiplex HYres Kit.

All experiments were performed with QIAGEN digital PCR mixes according to this invention or QIAGEN qPCR based DNA quant kits QIAGEN Investigator Quantiplex Pro Kit or QIAGEN Investigator Quantiplex HYres (QIAGEN, Hilden) in 20 μl reaction (total volume) following the manufacturer's instructions.

### FIGURE LEGENDS

FIG. 1: Precision Data Comparison Between the New and Present dPCR and the Prior Art qPCR DNA Quantification

FIG. 1: Precision data on human DNA quantification by digital PCR compared to state of the art qPCR. Shown are the DNA quantification data incl. mean values, standard deviation and coefficient of variation (CV) for digital PCR and two different state of the art qPCR quantification methods (QPro and QHYres). Noticeably, the method according to the invention (dPCR) demonstrates much higher precision compared to qPCR, in particular for the human target (Human) in digital PCR the CV is 1% compared to Human on qPCR where it is 7% for QPro and even 30% for QHyres. The difference is even higher for the Degradation target where the coefficient of variance is at 0.1% for digital PCR and 7% for QPro on qPCR, i.e. the degradation was detected more precisely by a factor of 70 compared to the method known in the art. These data clearly show a much higher precision and reliability for quantification of DNA with the invention. (na=not available). The coefficient of variation (CV) is defined as the ratio of the standard deviation divided by the mean. It shows the extent of variability in relation to the mean of the population. The lower the CV value the higher the precision for quantification and predicting the degree of degradation even if only single replicates of one sample are used.

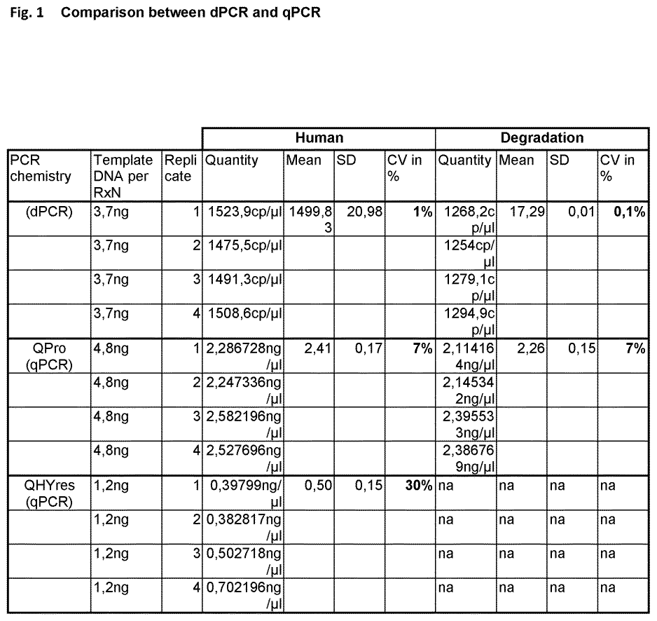

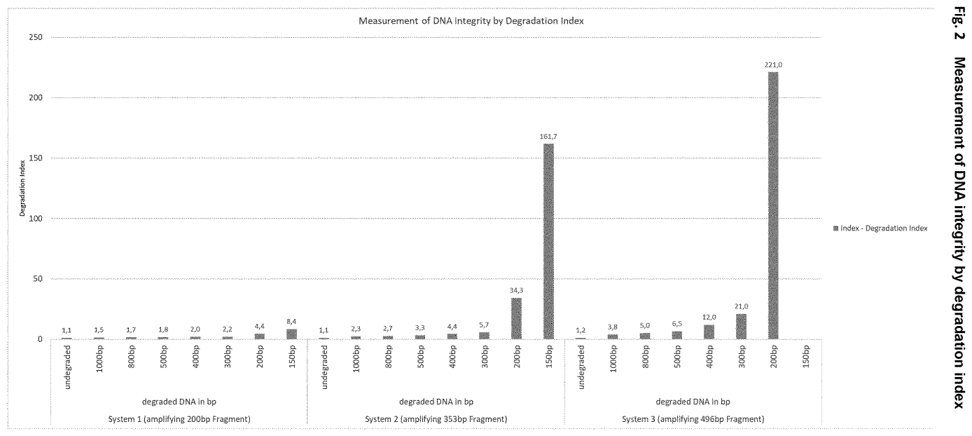

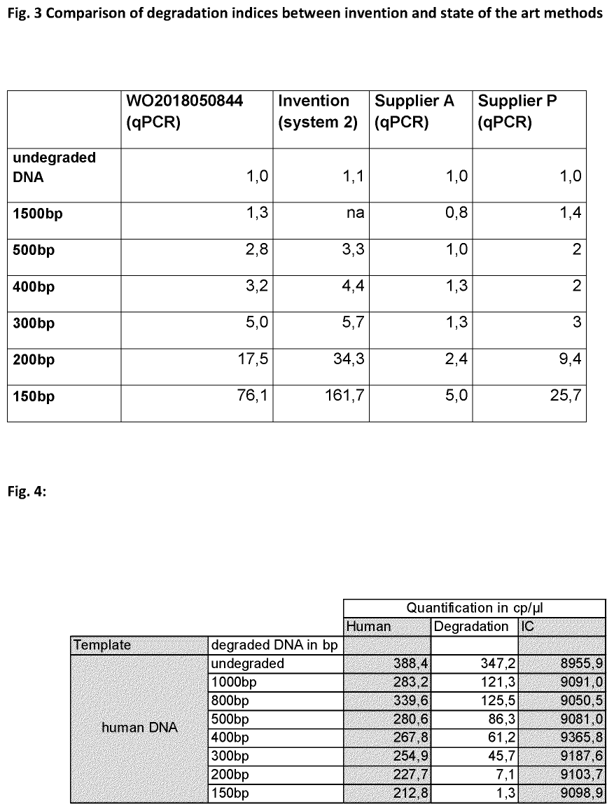

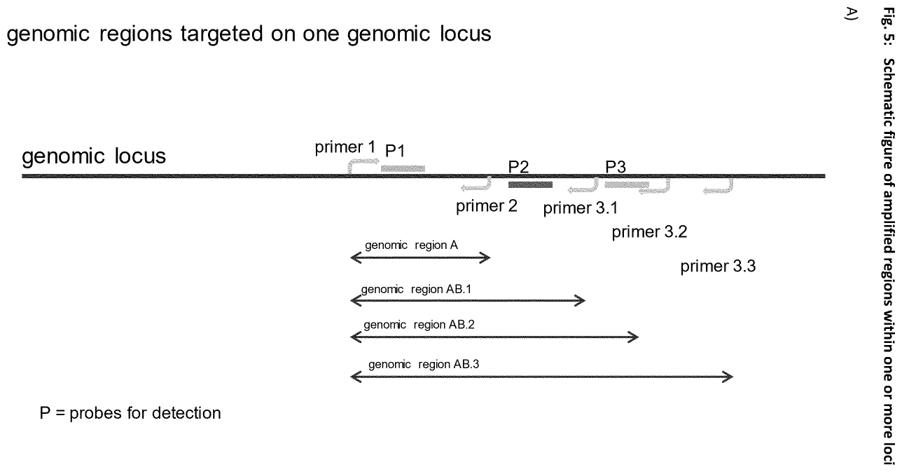

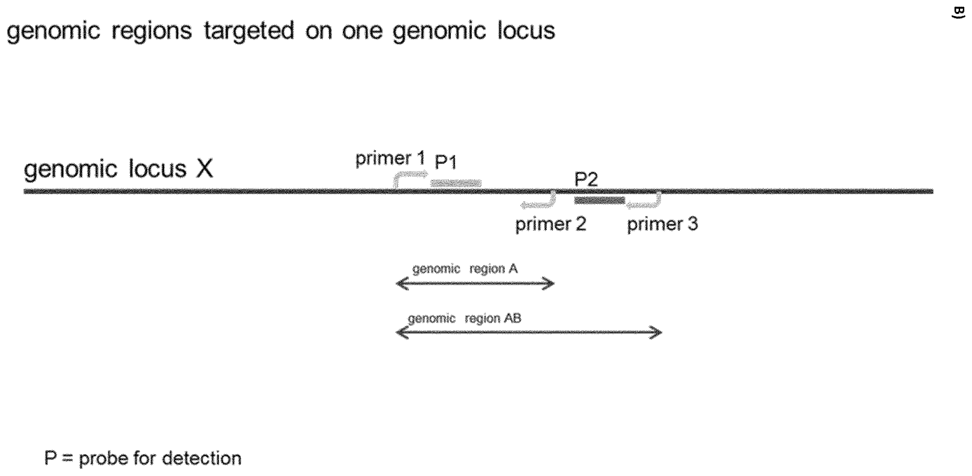

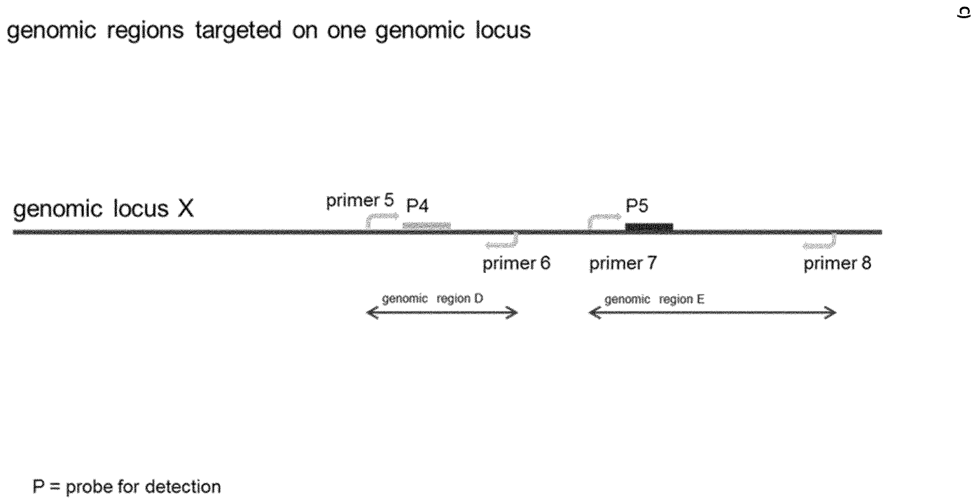

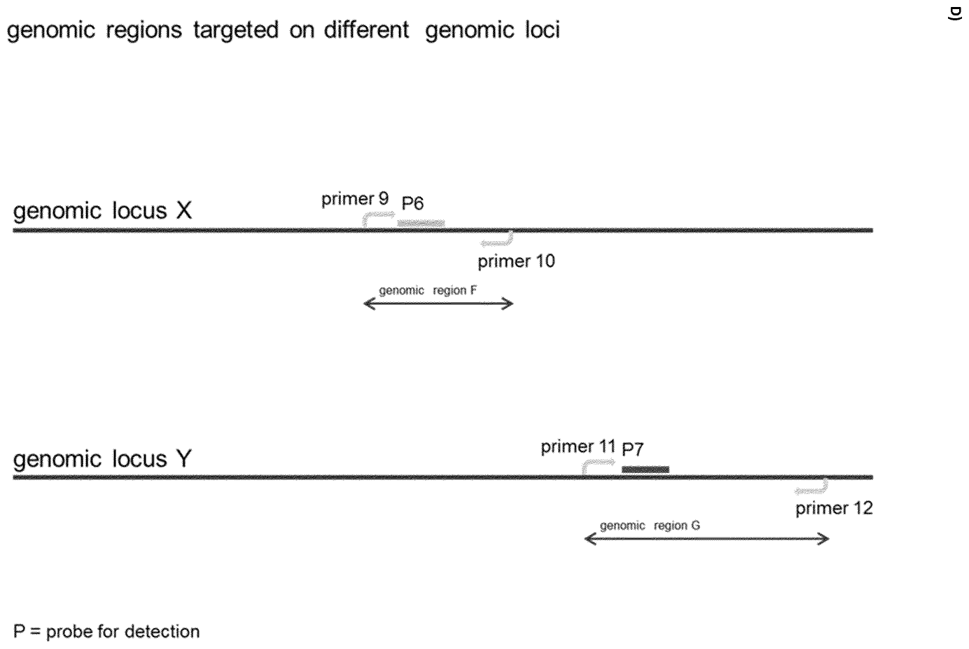

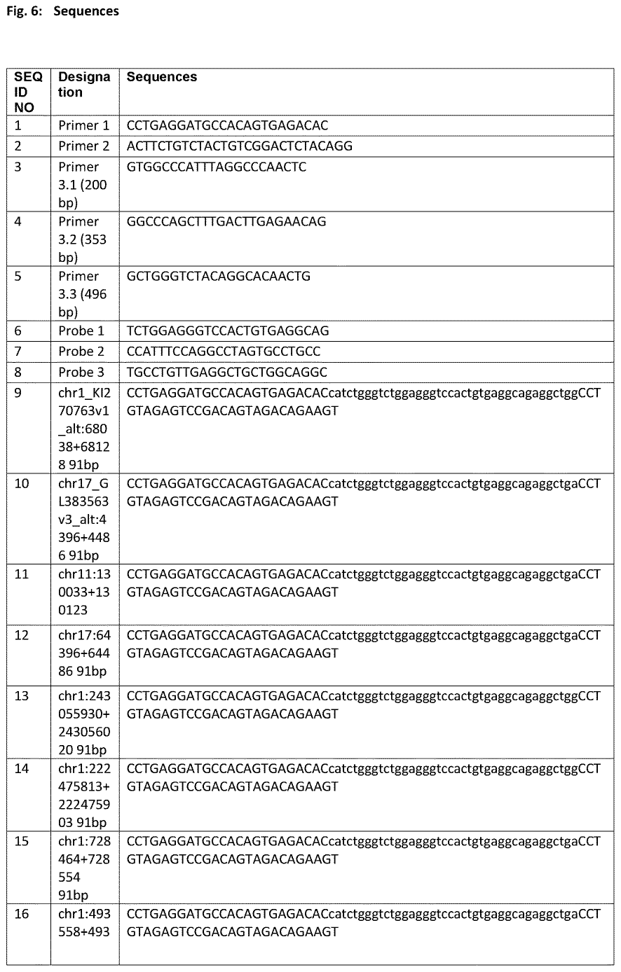

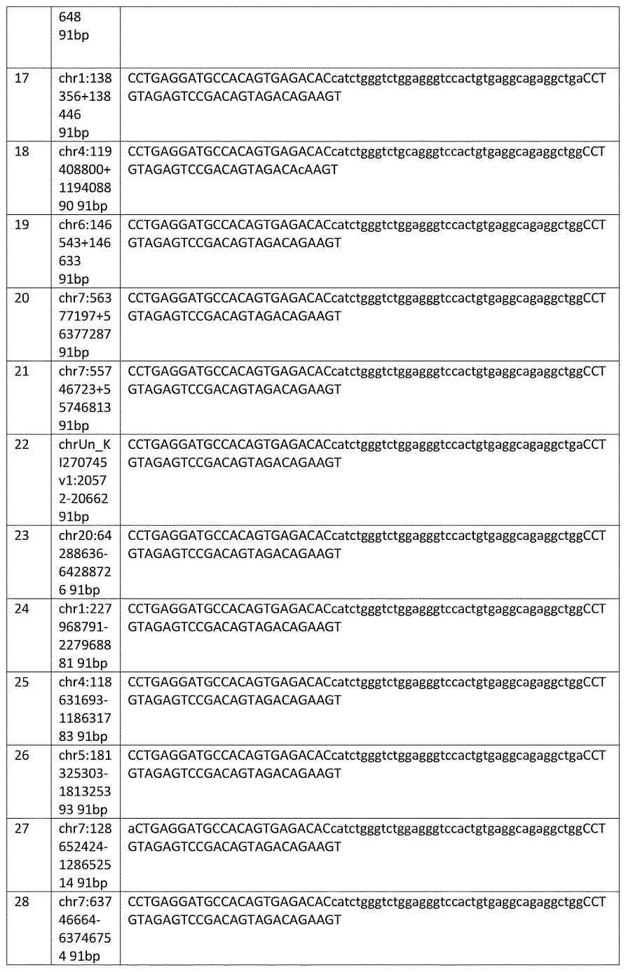

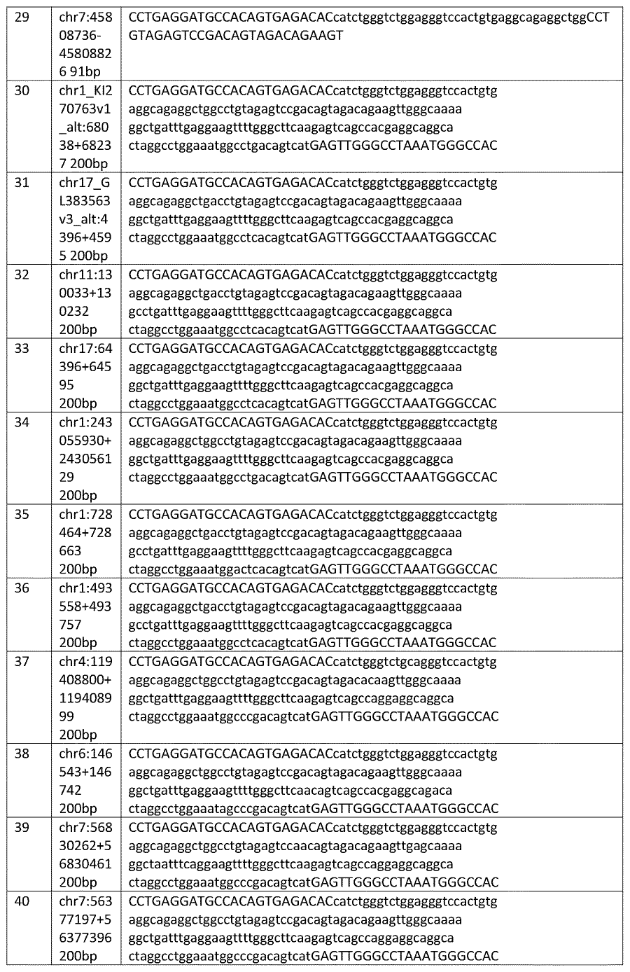

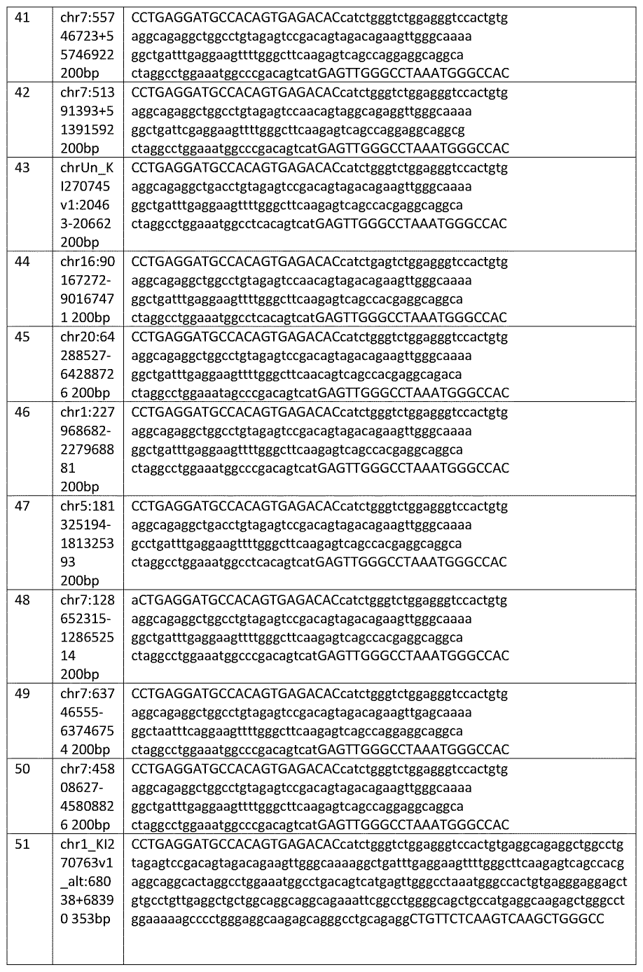

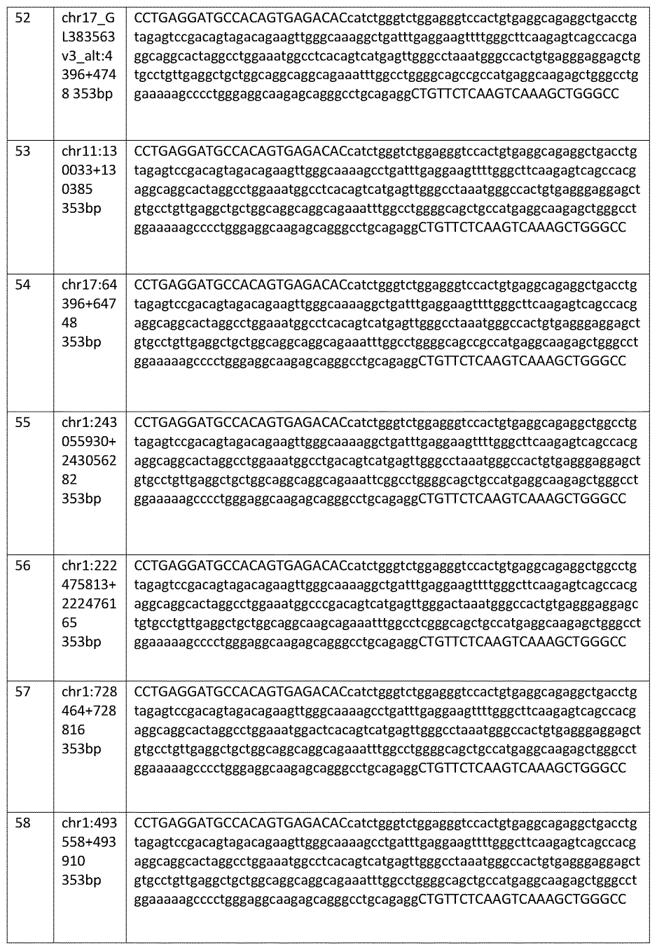

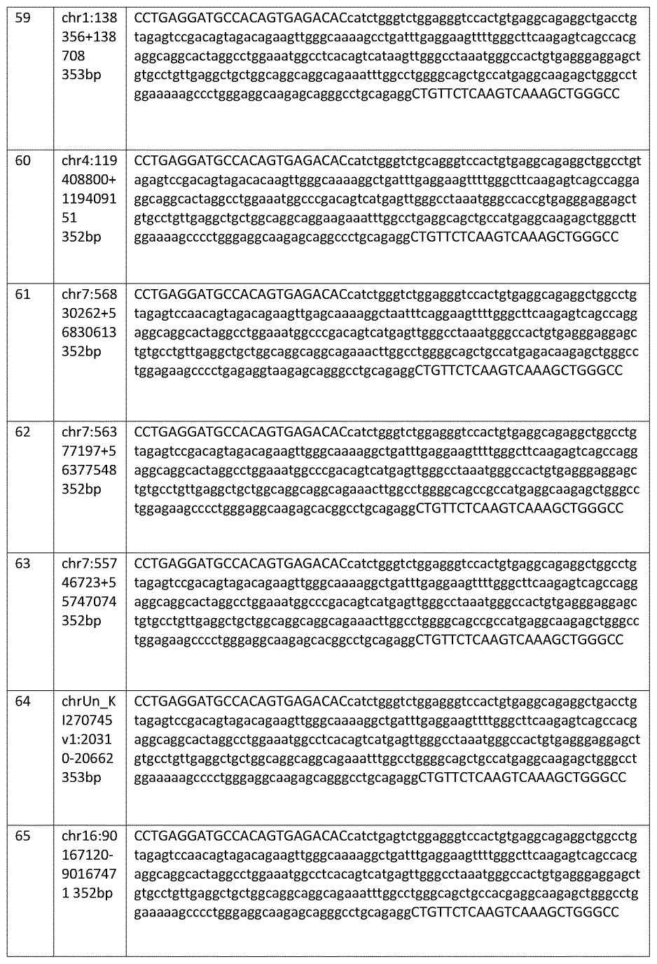

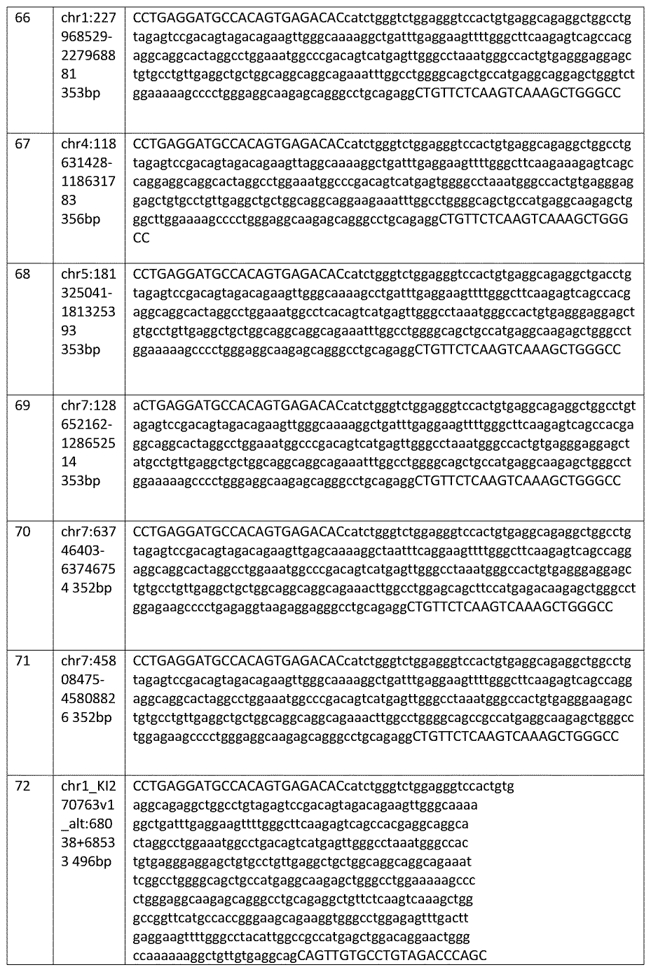

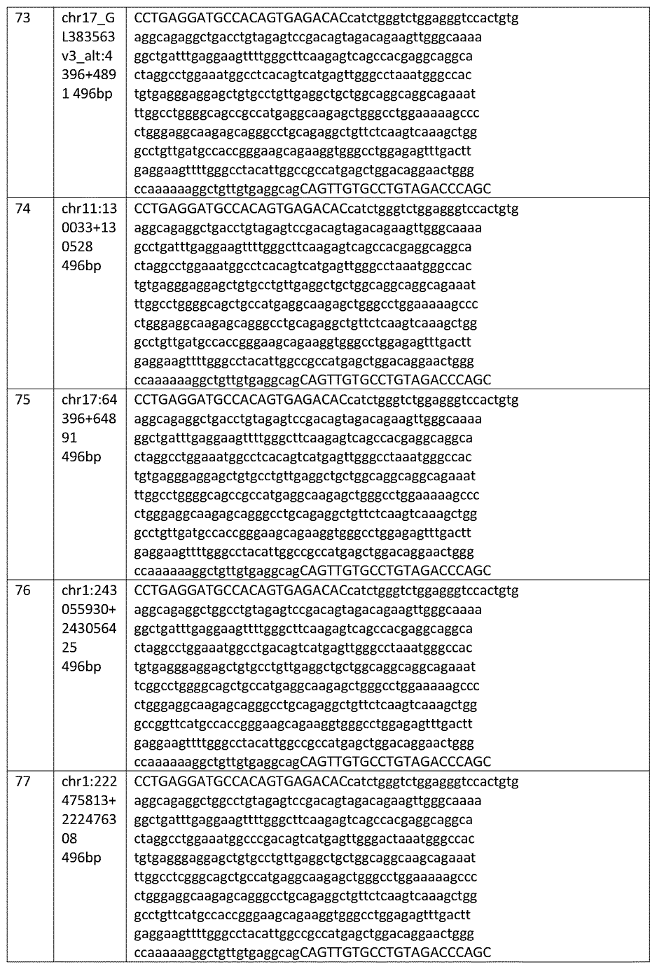

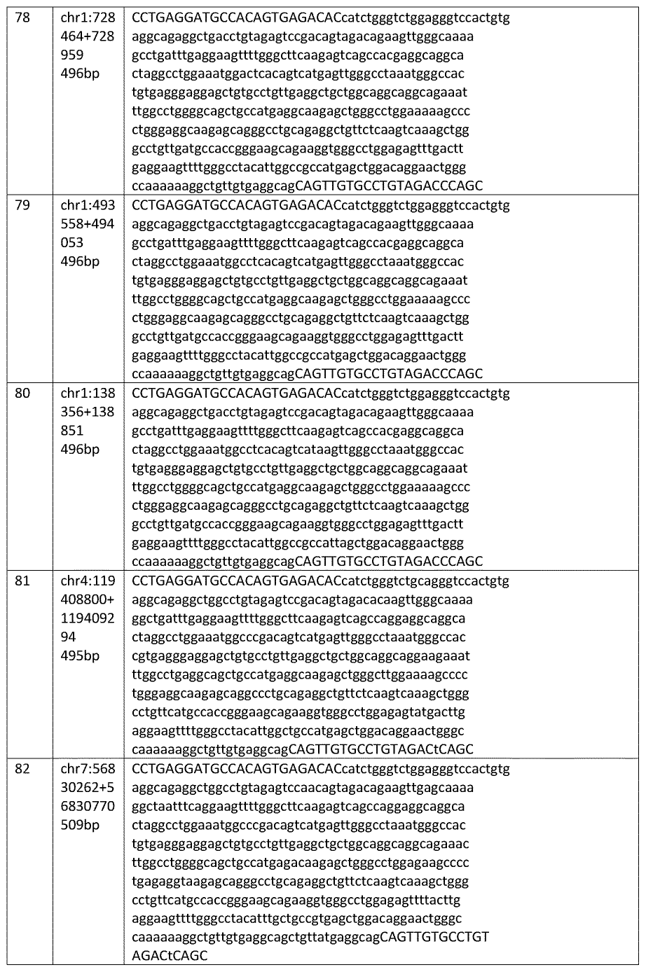

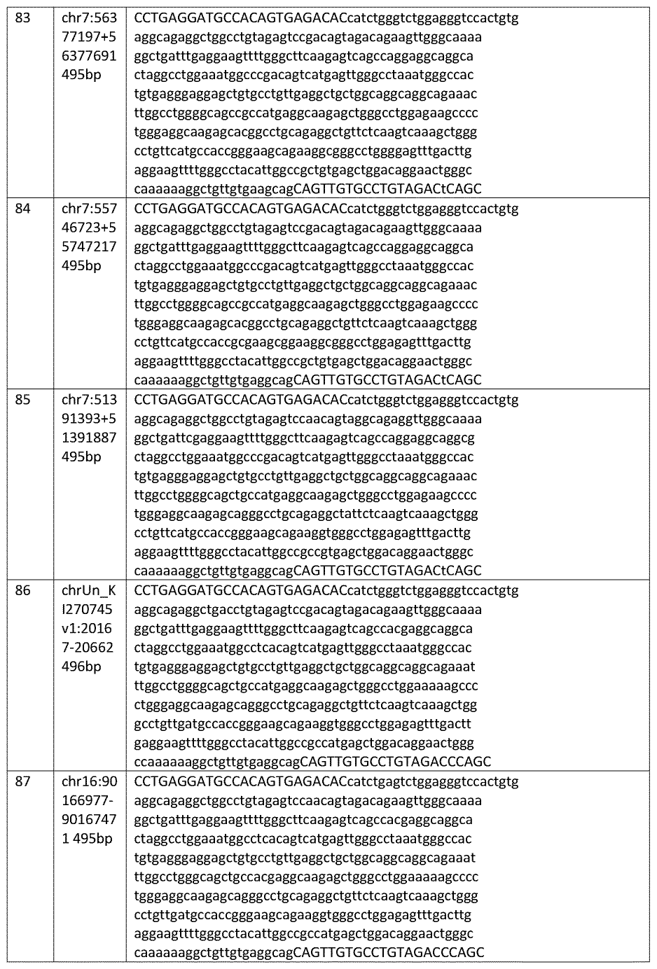

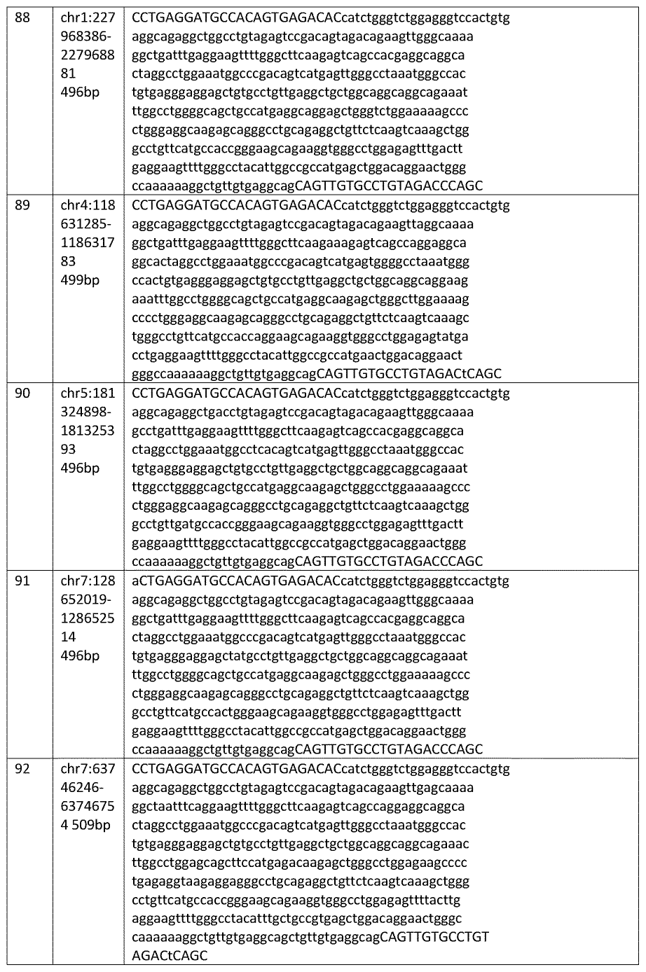

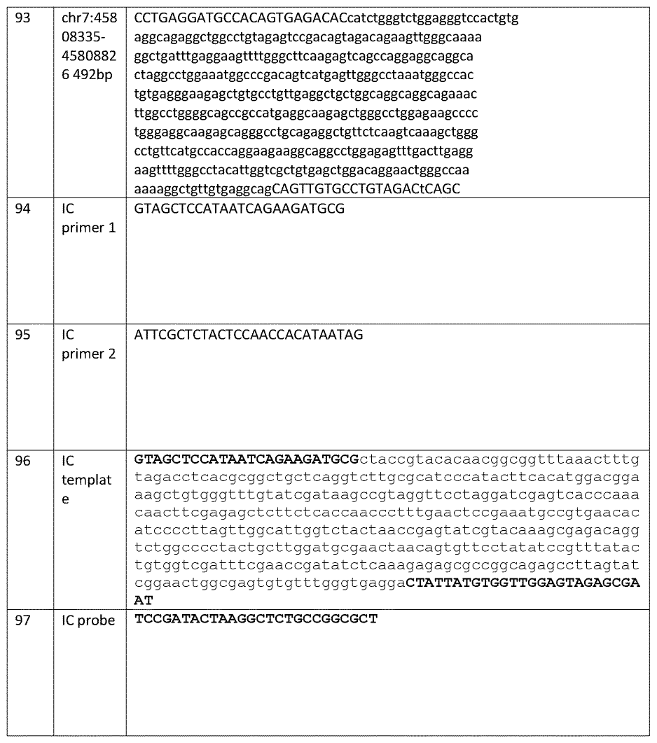

### FIG. 2: Measurement of DNA Integrity by Degradation Index

Measurement of DNA integrity by degradation index with three different amplicon sizes was performed.

Degraded human DNA with an average fragment length from 1000 bp, 800 bp, 500 bp, 400 bp, 300 bp, 200 bp and 150 bp has been applied to the method of invention. Degradation indices (DI) have been calculated by dividing the quantification value for human target (small target) by the quantification value of the degradation target (larger target). The DI value increases with higher degradation of the quantified DNA. Surprisingly the PCR system amplifying the 353 bp fragment generates DI with highest differences between degraded DNA fragments of 300 bp vs 200 bp vs 150 bp size. The expectation is that normally a larger amplicon size leads to a better resolution of the degradation state of template DNA. As shown in FIG. 2 the largest amplicon (496 bp) fails to detect the fragment size 150 bp of template DNA. The smallest fragment (200 bp) shows a significant lower increase of the Degradation Index over the whole range of degraded DNAs, making it more difficult to differentiate between the different degraded DNA fragments (e.g. fragment sizes of 300 bp to 250 bp). Therefore, the resolution using the 353 bp fragment is superior to a fragment of 200 bp as well as to a fragment of 496 bp. The table below is a numerical representation of the data shown in FIG. 2.

| | | Degradation | | Degradation |
|---|---|---|---|---|
| PCR Setup | Template DNA | State | | Index |
| System 1 | degraded DNA | undegraded | | 1.1 |
| (amplifying 200 | in bp | | | |
| bp Fragment) | | | | |
| | | 1000 | bp | 1.5 |
| | | 800 | bp | 1.7 |
| | | 500 | bp | 1.8 |
| | | 400 | bp | 2.0 |
| | | 300 | bp | 2.2 |
| | | 200 | bp | 4.4 |
| | | 150 | bp | 8.4 |
| System 2 | degraded DNA | undegraded | | 1.1 |
| (amplifying 353 | in bp | | | |
| bp Fragment) | | | | |
| | | 1000 | bp | 2.3 |
| | | 800 | bp | 2.7 |
| | | 500 | bp | 3.3 |
| | | 400 | bp | 4.4 |
| | | 300 | bp | 5.7 |
| | | 200 | bp | 34.3 |
| | | 150 | bp | 161.7 |
| System 3 | degraded DNA | undegraded | | 1.2 |
| (amplifying 496 | in bp | | | |
| bp Fragment) | | | | |
| | | 1000 | bp | 3.8 |
| | | 800 | bp | 5.0 |
| | | 500 | bp | 6.5 |
| | | 400 | bp | 12.0 |
| | | 300 | bp | 21.0 |
| | | 200 | bp | 221.0 |
| | | 150 | bp | |

The numbers in column 4 are the Degradation Index calculated by dividing the quantity of a smaller amplicon by the quantity of a larger amplicon. The more fragmented the template DNA the higher the degradation index because of a significant loss of possible template molecules for the larger amplicon. As an example, having a mean DNA size of 150 bp for a plurality of DNA molecules, wherein the size of the plurality of DNA molecules follows a Gaussian distribution, implies that only few molecules with a size of 353 bp and even less molecules having a size of 496 bp are present. Therefore, the degradation index allows a conclusion to the state of degradation of the unknown DNA in the customers sample.

### FIG. 3: Degradation Indices

Shown are the degradation indices (i.e. the ratio of the amount of short fragments vs. the amount of long fragments (human/degradation) of the different systems tested. Noticeably, the method according to the invention (second column) obtains consistently much higher indices, in particular for the smaller fragments ranging from 300 bp to 150 bp (a value of more than 160) compared to the other systems on qPCR. This indicates a much higher sensitivity/precision for the detection of degraded DNA and better resolution of DNA integrity. (na=has not been measured).

### FIG. 4: Multiplex Amplification of Human Target

Multiplex Amplification of human target, degradation target and Internal Amplification Control. A multiplex amplification setup according to the invention where three different targets (human (smaller amplification product), degradation (larger amplification product) and internal amplification control (IC)) have been successfully amplified in parallel. Noticeably, the method according is able to quantify human DNA, to assess DNA integrity and to report successful amplification by the use of an internal amplification control in parallel.

### FIG. 5: Schematic Figure of Amplified Locus

A) Four overlapping genomic regions targeted on one genomic locus. Depicted is a schematic drawing of the invention targeting different genomic regions on one genomic locus. The smaller PCR system comprises primer 1 and 2 and an appropriate probe for detection in dPCR and targets genomic region A. The larger PCR system comprises the same primer 1 and primer 3.1 or primer 3.2 or primer 3.3 extending the first genomic region A for the desired length and forming the second genomic region AB.1, AB.2 or AB.3, which harbours the whole genomic region A.
B) Two overlapping genomic regions targeted on one genomic locus. Depicted is a schematic drawing of the invention targeting different genomic regions on one genomic locus. The smaller PCR system comprises primer 1 and 2 and an appropriate probe for detection in dPCR and targets genomic region A. The larger PCR system comprises the same primer 1 and also primer 3 (SEQ ID NO. 4) extending the first genomic region A for the desired length and forming the second genomic region AB, which harbours the whole genomic region A.
C) Two non-overlapping genomic regions targeted on one genomic locus. Depicted is a schematic drawing of the invention targeting two genomic regions on one genomic locus, here a multi copy locus. The smaller PCR system comprises an appropriate primer/probe system for detection in dPCR and targets genomic region D. The larger PCR system comprises an appropriate primer/probe system for detection in dPCR and targets genomic region E which is a separate but adjacent genomic region to genomic region D. Genomic region E does not harbour or overlap with genomic region D.
D) Two non-overlapping genomic regions targeted on two genomic loci. Depicted is a schematic drawing of the invention depicting an approach where different genomic regions from different genomic loci are targeted. The smaller PCR system comprises an appropriate primer/probe system and targets genomic region F on genomic locus X. The larger PCR system comprises an appropriate primer/probe system that targets genomic region G on genomic locus Y.

### FIG. 6: Sequences

Shown are sequences for primers, probes and amplicons according to the invention. For amplicon sequences, the primer binding sites are indicated with capital letters.

1.-13. (canceled)

14. A method for assessment of the status of nucleic acid degradation and/or integrity of one or more nucleic acids in a sample, comprising the steps of:
amplifying at least two genomic regions within at least one genomic locus to produce at least two amplification products, wherein the amplifying is done by a digital PCR (dPCR) method; and
detecting the amount of the at least two amplification products through the use of at least two probes, wherein at least one of the at least two probes binds to one of the at least two amplification products and at least one other probe of the at least two probes binds to another one of the at least two amplification products.

15. The method according to claim 14, wherein the at least two genomic regions are overlapping and are amplified using at least one common primer.

16. The method according to claim 15, wherein at least one of the at least two probes binds to the region of overlap of said at least two amplification products and at least one other probe binds to a non-overlapping region of said at least two amplification products.

17. The method according to claim 14, wherein the size of the amplification products of the at least two genomic regions is between 20 base pairs and 2000 base pairs long.

18. The method according to claim 14, wherein a second one of the at least two amplification products is at least 30% longer than a first one of the at least two amplification products.

19. The method according to claim 18, wherein a third genomic region is amplified to produce a third amplification product, and wherein the third amplification product is at least 30% longer than the second amplification product.

20. The method according to claim 14, wherein the at least one genomic locus is a single copy locus (SCL) within the nucleic acid assessed for integrity and/or degradation.

21. The method according to claim 14, wherein the at least one genomic locus is a multicopy locus (MCL) within the nucleic acid assessed for integrity and/or degradation.

22. The method according to claim 14, wherein the method obtains a degradation index of at least 30 when measuring degraded DNA of 200 bp length and of at least 150 when measuring degraded DNA of 150 bp length.

23. The method according to claim 14, wherein in parallel to the assessment of the status of DNA degradation and/or integrity of one or more nucleic acids in a sample, the one or more nucleic acids are quantified and/or detected.

24. The method according to claim 14, wherein the sample is selected from the group consisting of human-, animal-, plant-, bacterial-, archaeal-, oomycetric, autosomal, human-autosomal or fungal DNA, environmental samples and food samples.

25. The method according to claim 14, wherein the dPCR method is a nanoplate based dPCR method.

26. The method according to claim 14, wherein the dPCR method is at least a triplex dPCR method, and wherein the amplified genomic regions comprise a human target, a degradation target and an internal amplification control region.

27. A kit comprising at least one of the primers and/or probes corresponding to SEQ ID NOs 1-8 or complements thereof for assessing the status of nucleic acid degradation and/or integrity of one or more nucleic acids using digital PCR amplification.

28. The kit according to claim 27 further comprising at least one of the primers and/or probes corresponding to SEQ ID NOs 94-95 and 97 or complements thereof.
